(19) United States
(12) Patent Application Publication
(10) Pub. No.: US 2025/0276924 A1
(43) Pub. Date: Sep. 4, 2025

(54) CHEMICAL AND BIOLOGICAL DEGRADATION OF ETHER PER- AND POLYFLUOROALKYL SUBSTANCES

(71) Applicant: The Regents of the University of California, Oakland, CA (US)

(72) Inventor: Yujie Men, Riverside, CA (US)

(21) Appl. No.: 19/067,763

(22) Filed: Feb. 28, 2025

Related U.S. Application Data
(60) Provisional application No. 63/560,530, filed on Mar. 1, 2024.

(51) Int. Cl. C02F 1/70; C02F 1/32; C02F 3/12; C02F 101/36; C07C 51/377; C12N 1/20

(52) U.S. Cl. CPC C02F 1/70; C02F 1/32; C02F 3/1205; C07C 51/377; C12N 1/20; C02F 2101/36; C02F 2301/046

(57) ABSTRACT

Methods of chemically and biologically degrading ether per- and polyfluoroalkyl substances (PFAS) are described. One example method of chemically and biologically degrading ether PFAS includes aerobic biodegradation with microorganisms and chemically degradation with UV light and sulfite. Examples of the method provide increased overall degradation of ether PFAS in wastewater.

### CROSS-REFERENCE TO RELATED APPLICATIONS

This application claims the benefit of U.S. Provisional Patent Application No. 63/560,530, filed on Mar. 1, 2024, which is incorporated herein by reference in its entirety.

### STATEMENT REGARDING FEDERALLY SPONSORED RESEARCH

This invention was made with government support under Contract No. W912HQ20C0033 awarded by the Department of Defense and Contract No. R01ES032668 awarded by the National Institute of Environmental Health Sciences.

### TECHNICAL FIELD

This patent document relates to biological environmental remediation, specifically wastewater decontamination.

### BACKGROUND

Per- and polyfluoroalkyl substances (PFAS) have attracted more public attention over the past several decades. PFAS-containing products have been extensively employed in diverse applications across commerce and industry. As research continuously demonstrates their persistence in the environment and bioaccumulation and toxicity in living organisms, many legacy PFAS, such as perfluorooctanoic acid (PFOA) and perfluorooctanesulfonic acid (PFOS), have been gradually phased out. In the meantime, fluoroalkylether substances (ether PFAS) have been designed and produced as alternative PFAS, including GenX [hexafluoropropylene oxide dimer acid (HFPO-DA)] and F-53B [6:2 chlorinated polyfluoroalkyl ether sulfonate (6:2 Cl-PFESA)]. Ether PFAS is one of the most important groups within the large PFAS family.

Notably, a variety of ether PFAS, including novel ones, have been detected in soil, wastewater, surface water, groundwater, and human blood. Consequently, understanding the environmental fate of ether PFAS is crucial for assessing their potential risks and developing effective mitigation strategies.

Although some studies have investigated the environmental occurrence and fate of some emerging ether PFAS, a significant knowledge gap remains regarding a systematic understanding of the biodegradability of various ether PFAS structures. Critical questions have not yet been tackled, such as which structures could be biotransformed and through what biotransformation pathways. More importantly, in addition to elucidating the fate of ether PFAS in microbially active environments, the long-standing unanswered question is whether microbial processes could be harnessed to enhance the relatively low chemical treatability of ether PFAS, thus reducing treatment costs.

### SUMMARY

One aspect of the disclosure provided herein relates to a method of chemically and biologically degrading ether per- and polyfluoroalkyl substance (PFAS) molecules, the method comprising depositing wastewater into a chemical treatment unit, wherein the chemical treatment unit contains one or more UV lamps and a sulfite solution, retaining the wastewater in the chemical treatment unit for a first time period, transferring the wastewater from the chemical treatment unit to a biological treatment unit, wherein the biological treatment unit contains a sludge community in aerobic conditions, retaining the wastewater in the biological treatment unit for a second time period, and testing the wastewater to determine the concentration of chemical compounds selected from the group consisting of ether PFAS and/or breakdown products from the biological degradation of ether PFAS compounds.

In some embodiments, the sulfite solution is generated from one or more of sodium sulfite and potassium sulfite. In some embodiments, the concentration of the sulfite solution is between about 10.0 mM and about 20.0 mM. In some embodiments, the concentration of the sulfite solution is about 10.0 mM.

In some embodiments, the one or more UV lamps emit UV radiation at approximate 254 nm.

In some embodiments, the sludge community is combined with an activated sludge bacterial community taken from a wastewater treatment plant. In some embodiments, the one or more bacteria are combined with an activated sludge bacterial community taken from a wastewater treatment plant. In some embodiments, the sludge community is an activated sludge community. In some embodiments, the sludge community is taken from a wastewater treatment plant.

In some embodiments, the method additionally comprises the steps transferring the wastewater into the chemical treatment unit from the biological treatment unit, retaining the wastewater in the chemical treatment unit for a third time period, transferring the wastewater from the chemical treatment unit to the biological treatment unit, and retaining the wastewater in the biological treatment unit for a fourth time period.

In some embodiments, the first time period is between about 24 hours to 48 hours. In some embodiments, the second time period is between about 5 days to about 15 days. In some embodiments, the second time period is about 8 days. In some embodiments, the third time period is between about 24 hours to 48 hours. In some embodiments, the fourth time period is between about 5 days to about 15 days. In some embodiments, the fourth time period is about 8 days.

In some embodiments, the biological treatment unit is inoculated with one or more bacteria. In some embodiments, the biological treatment unit is inoculated with a cell culture containing one or more bacteria.

In some embodiments, the chemical treatment unit and the biological treatment unit are connected in series. In some embodiments, the chemical treatment unit and the biological treatment unit are separate.

In some embodiments, the chemical treatment unit and/or the biological treatment unit are linked to a wastewater outlet flow from a commercial or industrial site. In some embodiments, the chemical treatment unit and/or the biological treatment unit are linked to a wastewater treatment plant. In some embodiments, the chemical treatment unit and/or the biological treatment unit are linked to a wastewater flow.

In some embodiments, the chemical treatment unit and/or the biological treatment unit contain a chemical monitoring system. In some embodiments, the wastewater contained within the chemical treatment unit or the biological treatment unit is tested for residual ether PFAS and degradation byproducts through automated sample removal and/or sample testing. In some embodiments, the wastewater contained within the chemical treatment unit or the biological treatment unit is tested for residual ether PFAS and degradation byproducts through manual sample removal and/or sample testing.

### BRIEF DESCRIPTION OF THE DRAWINGS

FIG. 1 shows the Expected and Unknown Met ID (Metabolite Identification) Workflow from Compound Discoverer 3.1 with the additional nodes Search Mass List, Search mzCloud, Search ChemSpider, Apply Spectral Distance, Apply mzLogic, and Map to Metabolika Pathways.

FIG. 2A shows the aerobic biotransformation and defluorination of ether PFAS E1.

FIG. 2B shows the aerobic biotransformation and defluorination of ether PFAS E2.

FIG. 2C shows the aerobic biotransformation and defluorination of ether PFAS E3.

FIG. 2D shows the aerobic biotransformation and defluorination of ether PFAS E4.

FIG. 2E shows the aerobic biotransformation and defluorination of ether PFAS E5.

FIG. 2F shows the aerobic biotransformation and defluorination of ether PFAS E6.

FIG. 2G shows the aerobic biotransformation and defluorination of ether PFAS E7.

FIG. 2H shows the aerobic biotransformation and defluorination of ether PFAS E8.

FIG. 2I shows the proposed aerobic biotransformation and defluorination pathways for E1, E5, and E7.

FIG. 3A shows the aerobic biotransformation and defluorination of ether PFAS E9.

FIG. 3B shows the aerobic biotransformation and defluorination of ether PFAS E10.

FIG. 3C shows the aerobic biotransformation and defluorination of ether PFAS E11.

FIG. 3D shows the aerobic biotransformation and defluorination of ether PFAS E12.

FIG. 3E shows the proposed aerobic biotransformation and defluorination pathways for E11 and E12.

FIG. 4A shows the fluoride formation in the sludge-only control with no addition of ether PFAS.

FIG. 4B shows the parent compound removal and fluoride ion formation in the heat-inactivated abiotic controls including both autoclaved sludge (AS) and autoclaved sludge filtrate (AF) for ether PFAS E1.

FIG. 4C shows the parent compound removal and fluoride ion formation in the heat-inactivated abiotic controls including both autoclaved sludge (AS) and autoclaved sludge filtrate (AF) for ether PFAS E2.

FIG. 4D shows the parent compound removal and fluoride ion formation in the heat-inactivated abiotic controls including both autoclaved sludge (AS) and autoclaved sludge filtrate (AF) for ether PFAS E4.

FIG. 4E shows the parent compound removal and fluoride ion formation in the heat-inactivated abiotic controls including both autoclaved sludge (AS) and autoclaved sludge filtrate (AF) for ether PFAS E5.

FIG. 4F shows the parent compound removal and fluoride ion formation in the heat-inactivated abiotic controls including both autoclaved sludge (AS) and autoclaved sludge filtrate (AF) for ether PFAS E6.

FIG. 4G shows the parent compound removal and fluoride ion formation in the heat-inactivated abiotic controls including both autoclaved sludge (AS) and autoclaved sludge filtrate (AF) for ether PFAS E7.

FIG. 4H shows the parent compound removal and fluoride ion formation in the heat-inactivated abiotic controls including both autoclaved sludge (AS) and autoclaved sludge filtrate (AF) for ether PFAS E8.

FIG. 4I shows the parent compound removal and fluoride ion formation in the heat-inactivated abiotic controls including both autoclaved sludge (AS) and autoclaved sludge filtrate (AF) for ether PFAS E11.

FIG. 4J shows the parent compound removal and fluoride ion formation in the heat-inactivated abiotic controls including both autoclaved sludge (AS) and autoclaved sludge filtrate (AF) for ether PFAS E12.

FIG. 5A shows the theoretical Gibbs free energy barriers of spontaneous defluorination of CF₃OH.

FIG. 5B shows the theoretical Gibbs free energy barriers of spontaneous defluorination of CF₃CF₂OH.

FIG. 6 shows the proposed aerobic biotransformation pathway of E6.

FIG. 7A shows the chromatograph of a TP of E12, TP273.

FIG. 7B shows the MS full-scan of a TP of E12, TP273.

FIG. 7C shows the MS² fragmentation profile of a TP of E12, TP273.

FIG. 8 shows the defluorination after eight days in the heat-inactivated abiotic control of the post-biological treatment for E2, E3, E8, E9, and E10.

FIG. 9A shows the chromatograph of a TP of GenX, TP372.

FIG. 9B shows the MS full-scan of a TP of GenX, TP372.

FIG. 9C shows the MS² fragmentation profile of a TP of GenX, TP372.

FIG. 9D shows the chromatograph of a TP of GenX, TP310.

FIG. 9E shows the MS full-scan of a TP of GenX, TP310.

FIG. 9F shows the MS² fragmentation profile of a TP of GenX, TP310.

FIG. 9G shows the chromatograph of a TP of GenX, TP224.

FIG. 9H shows the MS full-scan of a TP of GenX, TP224.

FIG. 9I shows the MS² fragmentation profile of a TP of GenX, TP224.

FIG. 9J shows the chromatograph of a TP of GenX, TP206.

FIG. 9K shows the MS full-scan of a TP of GenX, TP206.

FIG. 9L shows the MS² fragmentation profile of a TP of GenX, TP206.

FIG. 9M shows the chromatograph of a TP of GenX, TP142.

FIG. 9N shows the MS full-scan of a TP of GenX, TP142.

FIG. 9O shows the MS² fragmentation profile of a TP of GenX, TP142.

FIG. 10A shows the post-biological treatment of chemical treatment effluent of ether PFAS E2.

FIG. 10B shows the post-biological treatment of chemical treatment effluent of ether PFAS E3.

FIG. 10C shows the post-biological treatment of chemical treatment effluent of ether PFAS E8.

FIG. 10D shows the post-biological treatment of chemical treatment effluent of ether PFAS E9.

FIG. 10E shows the chromatograph of a TP of E2 and E3, TP222.

FIG. 10F shows the MS full-scan of a TP of E2 and E3, TP222.

FIG. 10G shows the MS² fragmentation profile of a TP of E2 and E3, TP222.

FIG. 10H shows the chromatograph of a TP of E2, E3, and E9, TP157.

FIG. 10I shows the MS full-scan of a TP of E2, E3, and E9, TP157.

FIG. 10J shows the MS² fragmentation profile of a TP of E2, E3, and E9, TP157.

FIG. 10K shows the chromatograph of a TP of E2 and E3, TP156.

FIG. 10L shows the MS full-scan of a TP of E2 and E3, TP156.

FIG. 10M shows the MS² fragmentation profile of a TP of E2 and E3, TP156.

FIG. 10N shows the chromatograph of a TP of E8, TP254.

FIG. 10O shows the MS full-scan of a TP of E8, TP254.

FIG. 10P shows the MS² fragmentation profile of a TP of E8, TP254.

FIG. 10Q shows the chromatograph of a TP of E8, TP236.

FIG. 10R shows the MS full-scan of a TP of E8, TP236.

FIG. 10S shows the MS² fragmentation profile of a TP of E8, TP236.

FIG. 10T shows the chromatograph of a TP of E8, TP175.

FIG. 10U shows the MS full-scan of a TP of E8, TP175.

FIG. 10V shows the chromatograph of a TP of E8, TP157.

FIG. 10W shows the MS full-scan of a TP of E8, TP157.

FIG. 10X shows the chromatograph of a TP of E9, TP340.

FIG. 10Y shows the MS full-scan of a TP of E9, TP340.

FIG. 10Z shows the MS² fragmentation profile of a TP of E9, TP340.

FIG. 10AA shows the chromatograph of a TP of E9, TP322.

FIG. 10BB shows the MS full-scan of a TP of E9, TP322.

FIG. 10CC shows the MS² fragmentation profile of a TP of E9, TP322.

FIG. 10DD shows the chromatograph of a TP of E9, TP260.

FIG. 10EE shows the MS full-scan of a TP of E9, TP260.

FIG. 10FF shows the MS² fragmentation profile of a TP of E9, TP260.

FIG. 10GG shows the chromatograph of a TP of E9, TP242.

FIG. 10HH shows the MS full-scan of a TP of E9, TP242.

FIG. 10II shows the MS² fragmentation profile of a TP of E9, TP242.

FIG. 10JJ shows the chromatograph of a TP of E9, TP228.

FIG. 10KK shows the MS full-scan of a TP of E9, TP228.

FIG. 10LL shows the MS² fragmentation profile of a TP of E9, TP228.

FIG. 10MM shows the chromatograph of a TP of E9, TP188.

FIG. 10NN shows the MS full-scan of a TP of E9, TP188.

FIG. 10OO shows the MS² fragmentation profile of a TP of E9, TP188.

FIG. 11 shows the defluorination degree of chemical (24-hr batch treatment) and aerobic biological post-treatment (8-day batch treatment) for five recalcitrant per- and polyfluorinated ether PFAS.

FIG. 12A shows the degradation of incomplete chemical defluorination products of GenX by aerobic biological post-treatment for GenX, TP162, TP144, TP140, TP127, and TP112.

FIG. 12B shows the degradation of incomplete chemical defluorination products of GenX by aerobic biological post-treatment for TP142, TP206, TP224, TP310, and TP372.

### DETAILED DESCRIPTION

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout the present specification and claims, the word “comprise” and variations thereof, such as, “comprises” and “comprising” are to be construed in an open, inclusive sense, that is, as “including, but not limited to.” As used herein, the terms “include” and “comprise” are used synonymously.

Unless specified otherwise, the terms “composition” and “composite” may be used interchangeably.

The disclosure provided herein aims to achieve cost-effective destruction of perfluorinated ether PFAS, such as GenX, using a chemical-biological treatment train system. In some embodiments, the chemical treatment unit consists of an advanced reduction process using hydrated electrons induced by UV/sulfite at pH 9.5. In some embodiments, the chemical treatment step can degrade GenX with about 40% total defluorination, resulting in incomplete reduced polyfluorinated products with C—H bonds. In some embodiments, the chemical treatment step is followed by an aerobic biological treatment step in a biological treatment unit. In some embodiments, the biological treatment step further converts the polyfluorinated products into short chain perfluorocarboxylic acids such as perfluoropropionic acid (PFPrA) and trifluoroacetic acid (TFA) with and additional about 20% defluorination. In some embodiments, the effluent is then recycled back into the chemical unit for further degradation of PFPrA and TFA. In some embodiments, the TFA is completely mineralized in the chemical unit. In some embodiments, the PFPrA is recycled back into the biological treatment unit for complete mineralization.

In comparison to advanced oxidation processes, which would result in the same short-chain perfluorinated end products, the biological post-treatment is more cost-effective, particularly when there is a high-level non-fluorinated dissolved organic carbon in the matrix that may severely affect the advanced oxidation process. For advanced oxidation processes, if high level dissolved organic carbon is present, more chemicals or energy would be needed to achieve the same removal efficiency of GenX. Thus, using aerobic biological processes would be less energy-consuming and more cost-effective than chemical oxidation. Existing biological wastewater treatment infrastructure may additionally be adapted for this purpose.

One aspect of the disclosure provided herein relates to a method of chemically and biologically degrading ether per- and polyfluoroalkyl substance (PFAS) molecules, the method comprising depositing wastewater into a chemical treatment unit, wherein the chemical treatment unit contains one or more UV lamps and a sulfite solution, retaining the wastewater in the chemical treatment unit for a first time period, transferring the wastewater from the chemical treatment unit to a biological treatment unit, wherein the biological treatment unit contains a sludge community in aerobic conditions, retaining the wastewater in the biological treatment unit for a second time period, and testing the wastewater to determine the concentration of chemical compounds selected from the group consisting of ether PFAS and/or breakdown products from the biological degradation of ether PFAS compounds.

In some embodiments, the wastewater was retained in the chemical treatment unit for a first time period ranging between about 24 to 48 hours.

In some embodiments, the wastewater was retained in the biological treatment unit for a second time period ranging between about 5 to 15 days. In some embodiments, the second time period was about 8 days.

In some embodiments, the wastewater was retained in the chemical treatment unit for a third time period ranging between about 24 to 48 hours.

In some embodiments, the wastewater was retained in the biological treatment unit for a fourth time period is about 8 days. In some embodiments, the fourth time period ranges from about 5 days to about 15 days.

In some embodiments, the sulfite solution has a concentration ranging from about 10 mM to about 20.0 mM.

In some embodiments, the sulfite solution is generated from one or more of sodium sulfite and potassium sulfite. In some embodiments, the concentration of the sulfite solution is about 10.0 mM.

In some embodiments, the one or more UV lamps emit UV radiation at approximately 254 nm.

In some embodiments, the sludge community is combined with an activated sludge bacterial community taken from a wastewater treatment plant. In some embodiments, the one or more bacteria are combined with an activated sludge bacterial community taken from a wastewater treatment plant.

In some embodiments, the method additionally comprises the steps transferring the filtered wastewater into the chemical treatment unit from the biological treatment unit, retaining the wastewater in the chemical treatment unit for a third time period, transferring the wastewater from the chemical treatment unit to the biological treatment unit, and retaining the wastewater in the biological treatment unit for a fourth time period.

In some embodiments, the first time period is between about 24 hours to 48 hours. In some embodiments, the second time period is between about 5 days to about 15 days. In some embodiments, the second time period is about 8 days. In some embodiments, the third time period is between about 24 hours to 48 hours. In some embodiments, the fourth time period is between about 5 days to about 15 days. In some embodiments, the fourth time period is about 8 days.

In some embodiments, the biological treatment unit is inoculated with a cell culture containing one or more bacteria.

In some embodiments, the chemical treatment unit and the biological treatment unit are connected in series. In some embodiments, the chemical treatment unit and the biological treatment unit are separate.

In some embodiments, the chemical treatment unit and/or the biological treatment unit are linked to a wastewater outlet flow from a commercial or industrial site. In some embodiments, the chemical treatment unit and/or the biological treatment unit are linked to a wastewater treatment plant. In some embodiments, the chemical treatment unit and/or the biological treatment unit are linked to a wastewater flow.

In some embodiments, the chemical treatment unit and/or the biological treatment unit contain a chemical monitoring system. In some embodiments, the wastewater contained within the chemical treatment unit or the biological treatment unit is tested for residual ether PFAS and degradation byproducts through automated sample removal and/or sample testing. In some embodiments, the wastewater contained within the chemical treatment unit or the biological treatment unit is tested for residual ether PFAS and degradation byproducts through manual sample removal and/or sample testing.

In some embodiments, the chemical treatment unit and/or the biological treatment unit contain a chemical monitoring system. In some embodiments, the chemical monitoring system may further comprise one or more analytical tools selected from the group consisting of an HPLC, HRMS, NMR, and a gas chromatograph.

### EXAMPLES

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein, to the extent that they do not contradict or are not inconsistent with the instant disclosure.

### Methods and Experimental Setup

### Photochemical Reactor Setup

The chemical treatment of five non-biodegradable PFAS was conducted in 600 mL closed-system batch reactors equipped with a low-pressure mercury lamp (254 nm, 18 W) at 20° C. The reaction mixture contained 25 μM ether PFAS, 10 mM Na₂SO₃, and 5 mM NaHCO₃ at pH 9.5 adjusted by NaOH. The reaction time was set up for 24 hours.

### Cell Pellets Extraction

The following cell pellet extraction method was based on the extraction method found in Che, S. et al. Environ. Sci. Technol. Lett. 8(8), 668-674 (2021). Two isotopic labelled PFAS, 13C-labeled PFBA (perfluoro-n-[2,3,4-13C3] butanoic acid) and 13C-labeled PFOA (perfluoro-n-octanoic acid-1-13C) were spiked in cell pellets as the surrogate with a final concentration of 20 μg/L (c.a. 92 nM and 48 nM for C13-PFBA and C13-PFOA, respectively) to account for extraction recovery. 1.5 mL methanol (with 1% NH₄OH) was then added to each vial, followed with a 20-minute vertexing. Each vial was then put into an ultrasonic bath for 45 minutes. Finally, the supernatant was collected after 20-minute centrifuge at 16,000 g at 4° C. for LC-HRMS/MS detection. The recovery of C13-PFBA and C-13PFOA were both over 90%.

### UHPLC-HRMS/MS Setup and Data Analysis

For the mobile phase of the UHPLC system, 10 mM ammonium acetate in Milli-Q water (A) and 10 mM ammonium acetate in HPLC grade methanol (B) were used. A two microliter sample was injected and eluted with a flow rate of 300 μL/min. The linear gradient was 95% A for 0-1 min, 95-5% A for 1-6 min, 5% A for 6-8.75 min, and 95% A for 8.75-12.5 min. For the deta-dependent MS², the normalized collision energy (NCE) was set at 25. The external calibration standards with matched matrix (i.e. sludge filtrate, 0.22 μm, for supernatant; methanol with 1% NH₄OH for cell extracts) were used for the quantification of targeted PFAS compounds. The peak areas of compounds were obtained by TraceFinder 4.1 EFS and Freestyle 1.8 (Thermo Fisher Scientific). The mass spectrometry was mainly obtained by Freestyle 1.8.

### Identification of TPs and Elucidation of Biotransformation Pathway

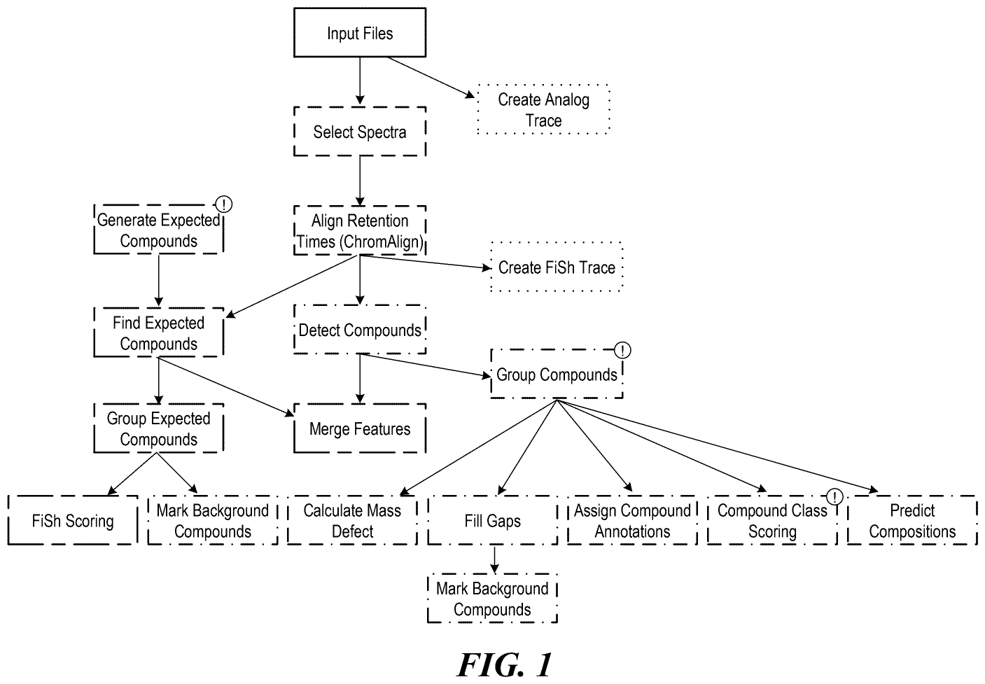

A custom-compiled suspect list was used to carry out suspect screening, which incorporated potential transformation products from biological reactions and pathways including monooxygenation, β-oxygenation, hydrogenation, dealkylation, and hydrolysis. Non-target screening was also applied by using a modified Expected and Unknown Met ID (Metabolite Identification) Workflow in Compound Discoverer 3.1 (Thermo Fisher Scientific) as shown in FIG. 1. The Expected and Unknown Met ID (Metabolite Identification) Workflow in Compound Discoverer 3.1 was modified with the following additional nodes: Search Mass List, Search mzCloud, Search ChemSpider, Apply Spectral Distance, Apply mzLogic, and Map to Metabolika pathways. To identify plausible TP's, the following criteria were employed: a mass tolerance of ≤5 ppm, an appropriate peak shape with a peak area exceeding 10⁵, an isotopic pattern score >70, a notable formation trend over time (initially increasing or increasing then subsequently decreasing), absence in abiotic, heat-inactivated, or sludge-only controls, and not recognized as in-source fragments. The structures of plausible TPs were clarified based on their MS² fragmentation profiles. For TPs with available reference compounds, structures were verified by comparing retention time (RT) and MS¹/MS² profiles between the TP and the reference compound. The criteria outlined by Charbonnet, J. A. et al. Environ. Sci. Technol. Lett. 9(6), 473-481 (2022) were used to determine TP confidence levels.

The most reasonable biotransformation pathways were suggested based on three aspects: the parent compound's removal trend, the structure/formula and formation trend of identified TPs, and the fluoride formation trend. Each of the three aspects should be consistent with one another. For example, in biotransformation pathways with multiple reaction stages, TPs from primary reactions should display a formation trend that first increases then decreases, succeeded by secondary TP transformation. In general, primary transient TPs reach their peak when the majority of the parent compound is depleted, after which the corresponding secondary TPs start to significantly rise, and this pattern continues for subsequent reaction stages. Stable products (or end products) exhibit an increasing trend, or an increasing trend followed by a plateau if the precursor is exhausted. The formation of fluoride should align with the formation of TPs containing fewer fluorine atoms.

### Theoretical Calculations

All the theoretical calculations were conducted by the Gaussian program (revision C.01). Geometrical structures of reactants, transition states and products were optimized by using the M06-2X functional, the Grimme's D3 empirical dispersion correction and the 6-311++G(3df,3pd) basis set (i.e., M06-2X-D3/6-311++G(3df,3pd)). The Gibbs free energy (G) was calculated by the following equation:

$$G = {E}^{SP} + {G}^{corr}$$

E^{SP} is the single-point energy refined by the coupled-cluster theory CCSD(T) implemented with the aug-cc-pVTZ basis set (i.e. CCSD(T)/aug-cc-pVTZ), while G^{corr} is the thermal corrections of Gibbs free estimated by frequency analysis with M06-2X-D3/6-311++G(3df,3pd). The SMD implicit solvent model was employed to simulate the water environment for all equations.

### Aerobic Biotransformation Experiments

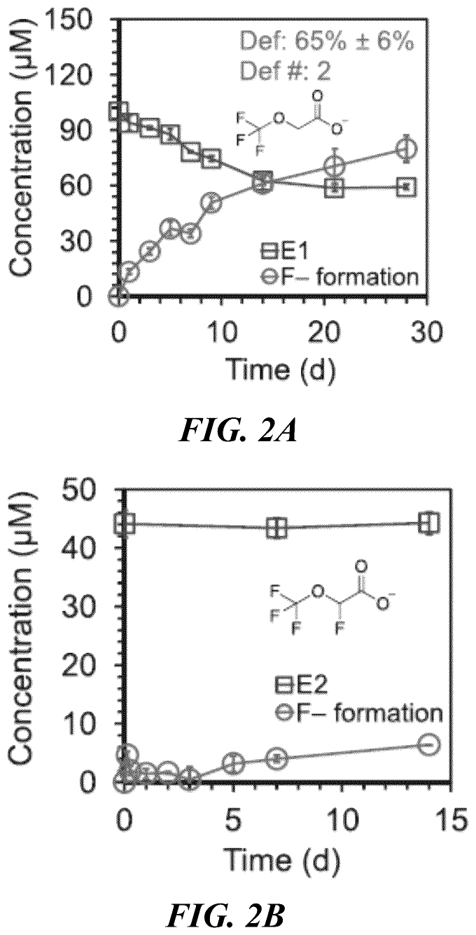

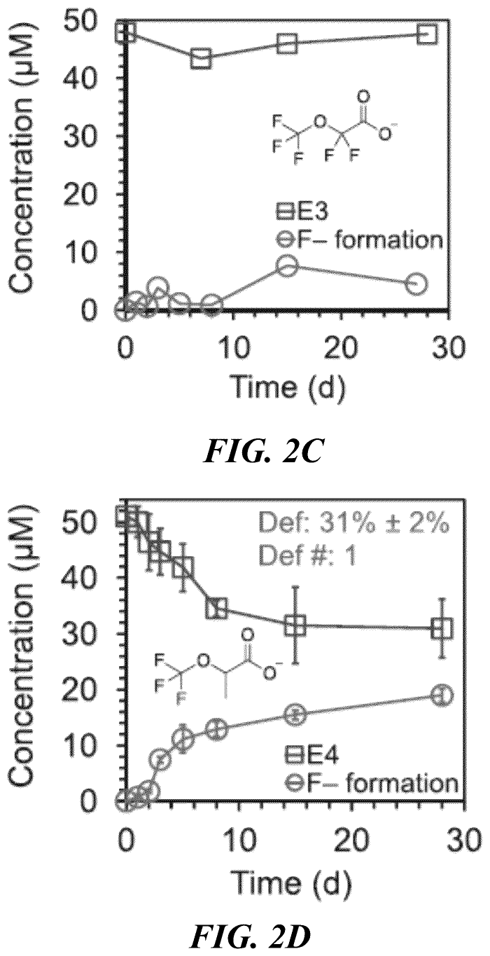

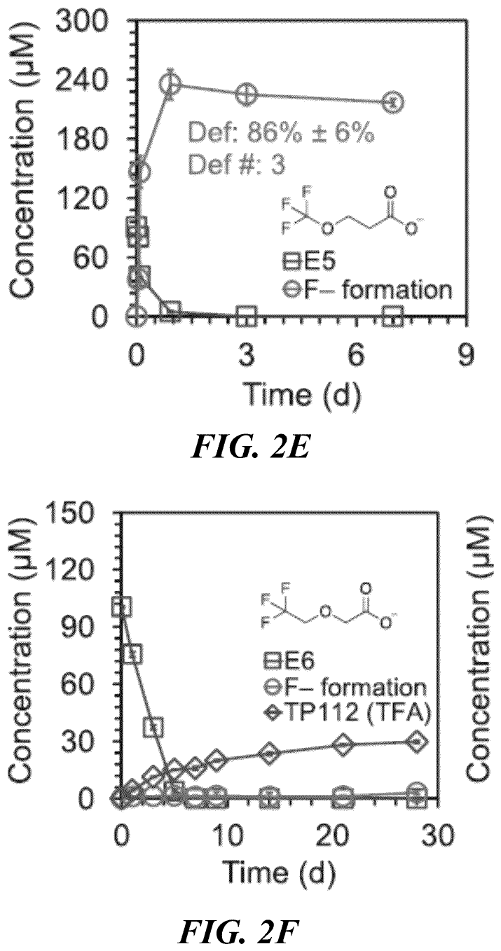

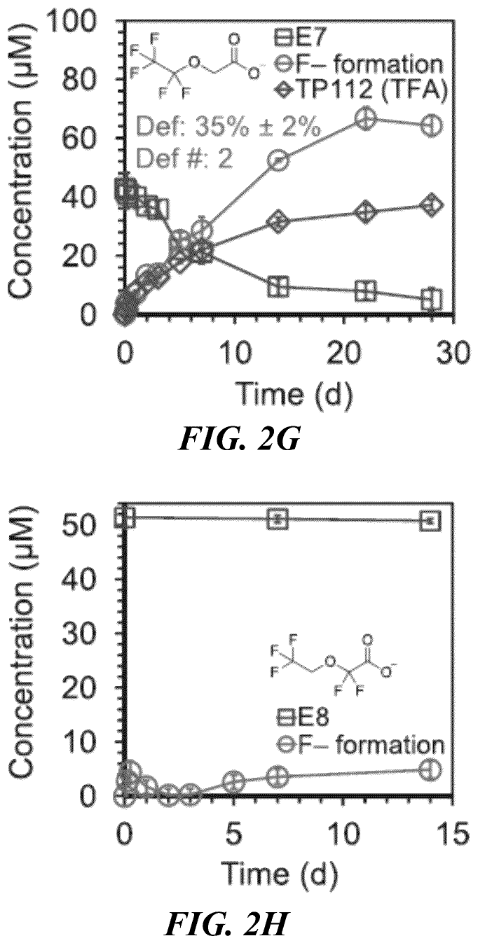

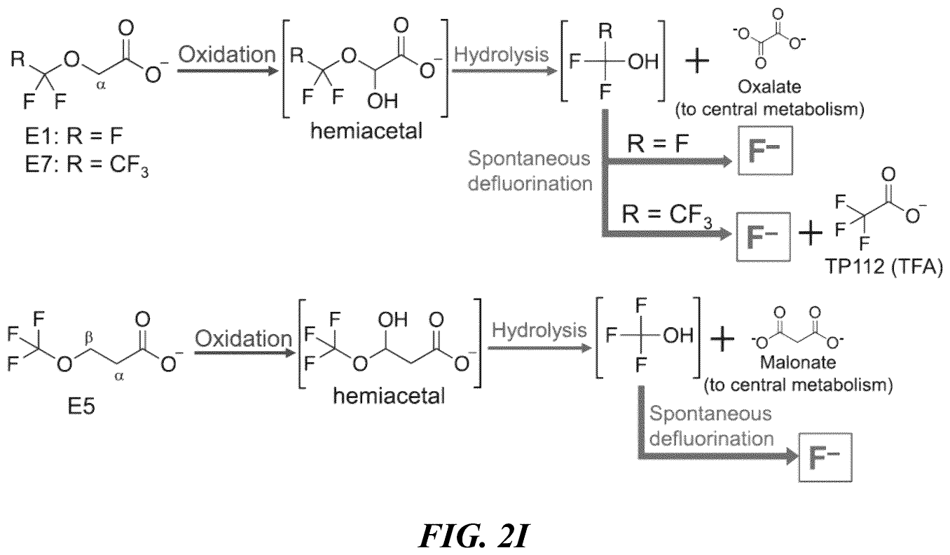

Twelve ether PFAS, as shown in FIGS. 2A-21, 3A-3E, and Table 1, were investigated, including eight structurally similar C₃-C₄ ether PFAS (E1-E8) and four C₅-C₆ structures, including GenX and its analogues (E9-E12). Some of them, such as GenX and E3, have been detected in aquatic environments, while some serve as building blocks for the synthesis of fluoropolymers and other fluorochemicals and may also be detected in the environment.

TABLE 1
Information for the investigated ether PFAS and confirmed transformation products (TPs).

| | ID | | Purity | RT | LOD | LOQ |
|---|---|---|---|---|---|---|
| Compound* | No .** | Structure (Formula) | (%) | (min) | (mM) | (mM) |
| 2-(trifluoromethoxy)acetic acid⁽¹⁾ | E1 | 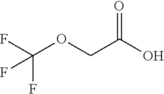 | 97 | 1.49 | 0.3 | 1 |
| | | C₃H₃F₃O₃ | | | | |
| 2-fluoro-2- (trifluoromethoxy)acetic acid⁽²⁾ | E2 | 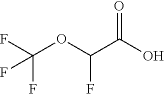 | 95 | 1.88 | 0.05 | 0.1 |
| | | C₃H₂F₄O₃ | | | | |
| 2,2-difluoro-2- (trifluoromethoxy)acetic acid⁽³⁾ | E3 | 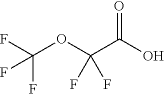 [SMILES: O=C(O)C(F)(F)OC(F)(F)F] | 98 | 3.67 | 0.05 | 0.1 |
| | | C₃HF₅O₃ | | | | |
| 2-(trifluoromethoxy)propanoic acid⁽⁴⁾ | E4 | 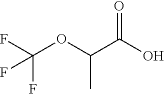 | 95 | 2.25 | 0.3 | 0.5 |
| | | C₄H₅F₃O₃ | | | | |
| 3-(trifluoromethoxy)propanoic acid⁽⁴⁾ | E5 | 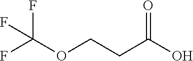 | 95 | 1.97 | 5 | 10 |
| | | C₄H₅F₃O₃ | | | | |
| 2-(2,2,2-trifluoroethoxy)acetic acid⁽⁵⁾ | E6 | 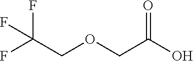 | 98 | 1.60 | 0.05 | 0.1 |
| | | C₄H₅F₃O₃ | | | | |
| 2-(perfluoroethoxy)acetic acid⁽⁴⁾ | E7 | 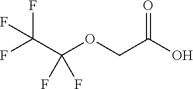 [SMILES: O=C(O)COC(F)(F)C(F)(F)F] | 97 | 4.44 | 0.05 | 0.3 |
| | | C₄H₃F₅O₃ | | | | |
| 2,2-difluoro-2-(2,2,2- trifluoroethoxy)acetic acid⁽⁵⁾ | E8 | 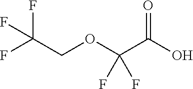 [SMILES: O=C(O)C(F)(F)OCC(F)(F)F] | 95 | 2.78 | 0.01 | 0.05 |
| | | C₄H₃F₅O₃ | | | | |
| 2,2,3,3,4,4-hexafluoro-4- (trifluoromethoxy)butanoic acid⁽¹⁾ | E9 | 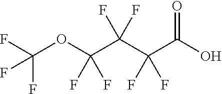 [SMILES: O=C(O)C(F)(F)C(F)(F)C(F)(F)OC(F)(F)F] | 97 | 6.33 | 0.01 | 0.05 |
| | | C₅HF₉O₃ | | | | |
| 2,3,3,3-tetrafluoro-2- (perfluoropropoxy)propanoic acid⁽¹⁾ | E10 (GenX) | 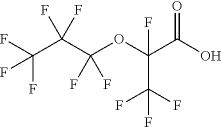 [SMILES: O=C(O)C(F)(OC(F)(F)C(F)(F)C(F)(F)F)C(F)(F)F] | 97 | 6.73 | 0.3 | 0.5 |
| | | C₆HF₁₁O₃ | | | | |
| 4,5,5,5-tetrafluoro-4- (trifluoromethoxy)pentanoic acid⁽⁴⁾ | E11 | 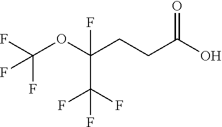 [SMILES: O=C(O)CCC(F)(OC(F)(F)F)C(F)(F)F] | 95 | 6.39 | 0.1 | 0.5 |
| | | C₆H₅F₇O₃ | | | | |
| 4,5,5,5-tetrafluoro-4- (trifluoromethoxy)pent-2-enoic acid⁽¹⁾ | E12 | 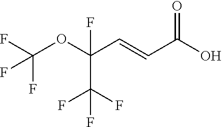 [SMILES: O=C(O)/C=C/C(F)(OC(F)(F)F)C(F)(F)F] | 95 | 6.41 | 0.01 | 0.05 |
| | | C₃H₃F₇O₃ | | | | |
| Trifluoroacetic acid⁽¹⁾ | TP112 (TFA) | 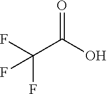 | 99 | 1.10 | 0.01 | 0.05 |
| | | C₂HF₃O₂ | | | | |
| 2,2,3,3,3-pentafluoropropanoic acid⁽¹⁾ | TP162 (PRPrA) | 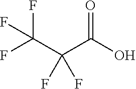 | 98 | 2.22 | 0.001 | 0.05 |
| | | C₃HF₅O₂ | | | | |
| 2,3,3,3-tetrafluoropropanoic acid⁽¹⁾ | TP144 | 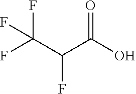 | 97 | 1.23 | 0.005 | 0.01 |
| | | C₃H₂F₄O₂ | | | | |
| 3,3,3-trifluoropropanoic acid⁽¹⁾ | TP127 | 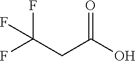 | 97 | 1.14 | 0.05 | 0.5 |
| | | C₃H₃F₃O₂ | | | | |
| Trifluoropyruvate | TP140 | 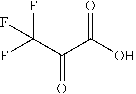 | 97 | 1.03 | 0.05 | 0.1 |
| | | C₃HF₃O₃ | | | | |

*Ether PFAS compounds were purchased from five companies:
⁽¹⁾SynQuest Laboratories Inc (Alachua, FL, USA);
⁽²⁾ChemSpace US Inc (Monmouth Junction, NJ, USA);
⁽³⁾Fluoryx Labs (Carson City, NV, USA);
⁽⁴⁾Manchester Organics Ltd (Runcorn, UK);
⁽⁴⁾abcr GmbH (Karlsruhe, Germany);
⁽⁵⁾Accela ChemBio Inc. (San Diego, CA, USA).
LOD: limit of detection determined as the lowest concentration with a signal-to-noise ratio of 30 or above;
LOQ: limit of quantification determined as the lowest concentration with a quantification error withing ±20%

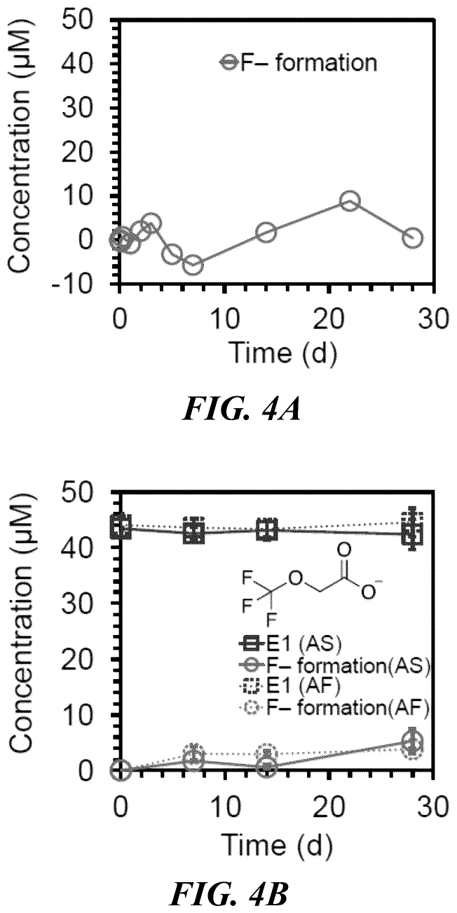

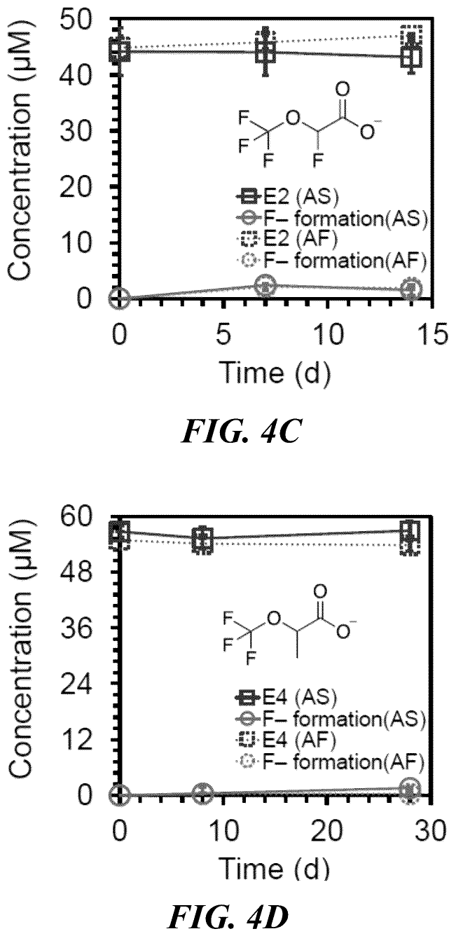

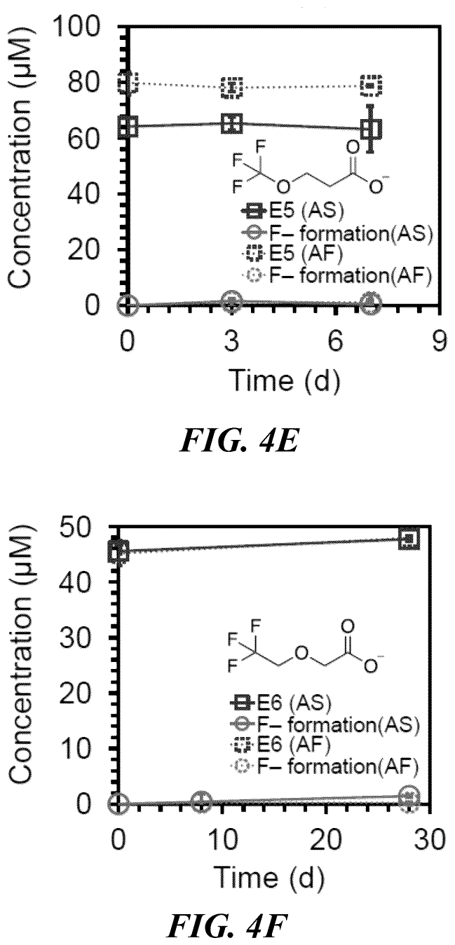

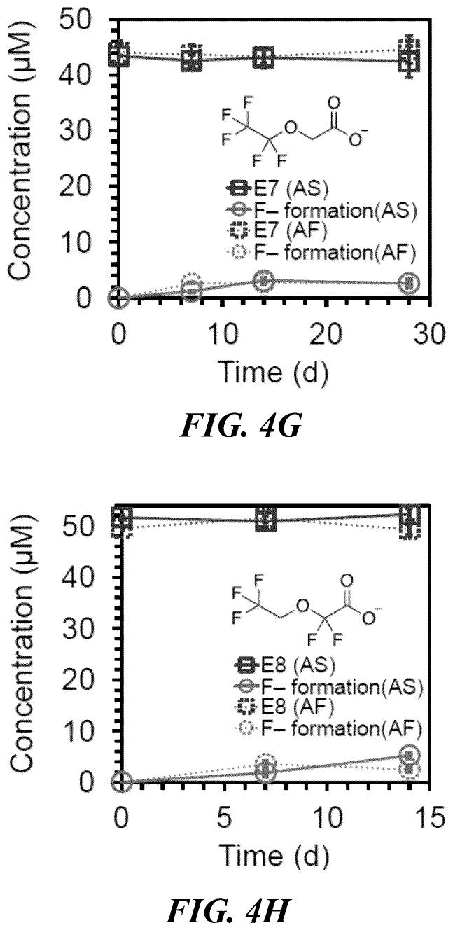

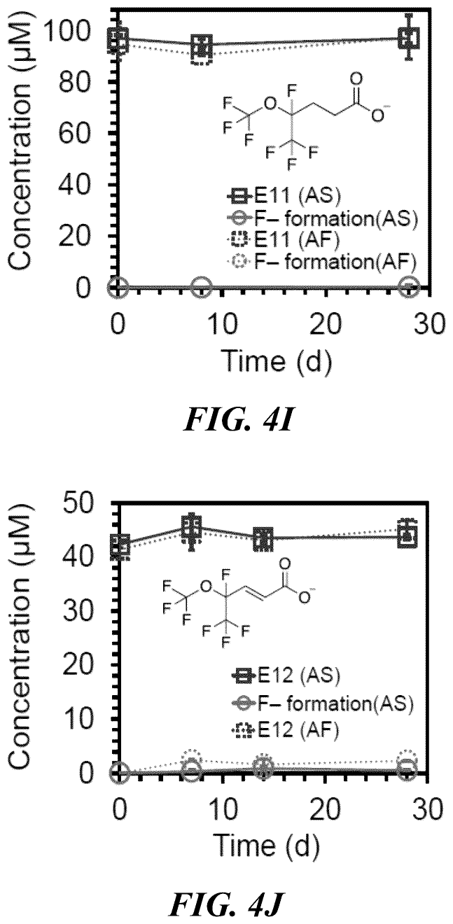

50 mL of an activated sludge community freshly taken from a local municipal wastewater treatment plant (WWTP), about 4400 mg/L as total suspended solids, was added to each 150 mL loosely capped batch reactor spiked with individual ether PFAS at an initial concentration of 50 μM except for E1, E5, E6, and E11, whose initial concentration was 100 μM due to their detection limit being higher than the other tested ether PFAS. The initial 100 μM allowed for more accurate analysis of the parent compound and transformation products. Reactors were incubated at room temperature for ≤28 days on a shaker at 150 rpm with a dissolved oxygen level of >3 mg/L and incubated with a Hach DO probe. Methanol and ammonium were added as the supplemental carbon and nitrogen source respectively to each reactor to sustain the sludge activity after three days in the same method as found in Che, S. et al. Environ. Sci. Technol. Lett. 8, 668-674 (2021) and Yu, Y. et al. Environ. Sci. Technol. 56, 4894-4904 (2022). Abiotic controls were also performed in the same setup, which included sludge autoclaved at 121° C. for 40 minutes and 0.22 μm autoclaved sludge filtrate. The sludge-only controls without the spike of ether PFAS were also set up to account for the fluorine ion concentration in the sludge matrix, where the level of fluorine ions fluctuated, with a maximum formation of about 10 μM during the incubation period as shown in FIG. 4A. Thus, only the fluorine ion levels greater than 10 μM were considered in the experimental samples as active defluorination.

For the integrated chemical and biological transformation experiments, 25 mL of the effluent from the advanced reduction treatment using hydrated electrons in ultraviolet (UV)/sulfite reactors was added to 25 mL of a two-fold concentrated activated sludge community in the 150 mL batch reactors with the same incubation conditions, as seen in Bentel, M. et al. Environ. Sci. Technol. 54(4), 2489-2499 (2020). The two-fold activated sludge community was generated by removing 25 mL of the supernatant after settling down 50 mL of sludge.

About 1.5 mL of samples were taken at multiple time points during the aforementioned biotransformation experiments and centrifuged at 13,000 rpm for 20 min. About 1.2 mL of the supernatant was collected to measure the parent compound and transformation products (TPs). The cell-associated parent compound and TPs were extracted using 1.5 mL of methanol with 1% NH₄OH. The samples were stored at 4° C. and analyzed within three weeks.

### Fluoride Measurement

The HQ30D Portable Multi Meter (HACH) connected with an ion-selective electrode (ISE, HACH) was used for the fluoride ion measurement. Fluoride Ionic Strength Adjustor (ISA) powder was added to eliminate the interference from aluminum and iron in the matrix before detection. The limit of quantification (LOQ) was 0.01 mg/L, about 0.5 μM. The fluoride measurement in the same matrix was cross-validated using ion chromatography in the same method as in Che, S. et al. Environ. Sci. Technol. Lett. 8, 668-674 (2021). The defluorination degree (Def %) was calculated with the equation below:

$$Def \% = \frac{maximum {F}^{-} formation ( nM )}{removed concentration ( mM ) x no . of F in one molecule} \times 100 \%$$

### Analytical Methods

An ultra-high-performance liquid chromatograph coupled with a high-resolution mass spectrometer (UHPLC-HRMS/MS, Q Exactive, Thermo Fisher Scientific) was employed for the detection of parent compounds and TPs. A hypersil Gold Column (particle size of 1.9 μm, 2.1 mm×100 mm, Thermo Fisher Scientific) was used for UHPLC separation. For HRMS detection, the negative electrospray ionization mode (ESI⁻) was used with a resolution of 70,000 @ m/z 200 for the full scan (m/z 70-1050) and 17,500 @ m/z 200 for the data-dependent MS² scan. For TP identification, both suspect and nontarget screening were applied.

### Example 1

### Substrate Specificity of Aerobic Biotransformation and Defluorination of Ether PFAS

For short-chain ether PFAS, between C3-C4, the aerobic biotransformation was observed for only those with at least one nonfluorinated carbon between the ether and the carboxyl group, such as E1 and E4-E7 as shown in FIGS. 2A-2H and FIGS. 4A-4J. This observation is consistent with findings in the literature where active aerobic biotransformation of short-chain polyfluorocarboxylic acids (PFCAs) required nonfluorinated carbon next to the carboxylic group. It has been reported that 2H-3:2PFECA (C₃F₇OCHFCOO⁻) with a monofluorinated (—CHF—) moiety next to the ether bond also exhibited slow aerobic biotransformation via a similar O-dealkylation pathway with a small amount of perfluoropropionic acid (PFPrA) being formed. This is consistent with observations that E2, which contains the same —CHF— moiety, did not exhibit notable parent compound decay or defluorination during a shorter incubation period.

For the five biotransformed ether PFAS structures, the aerobic biotransformation was initiated by the oxidation (i.e. hydroxylation) at the nonfluorinated carbon set next to the ether group, forming an unstable hemiacetal intermediate, which spontaneously rearranges into an alcohol product and an aldehyde product. This pathway of ether scission has been observed in various microorganisms according to detection of the expected end products. It could be catalyzed by monooxygenases and has been observed in the aerobic biodegradation of dialkyl ethers, 1,4-dioxane, and chlorinated dialkyl ethers such as bis(2-chloroethyl) ether. The aldehyde product from the hemiacetal group can be further oxidized, forming a carboxyl group. The non-fluorinated dicarboxylic acid products such as oxalate from E1 and malonate from E5 can enter the central metabolic pathway.

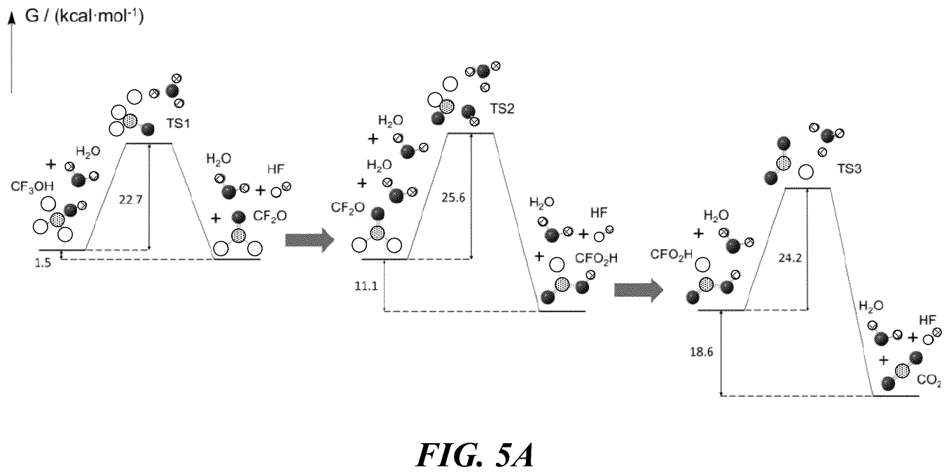

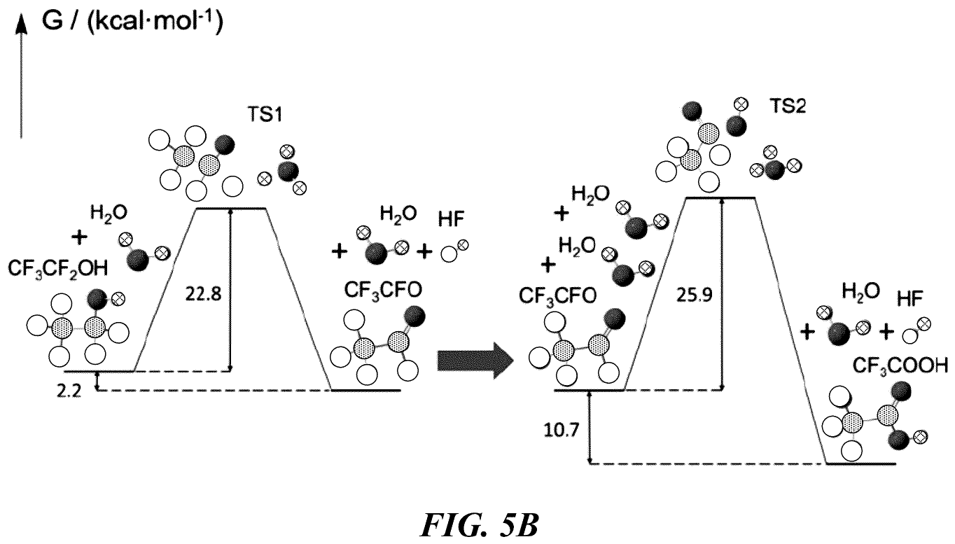

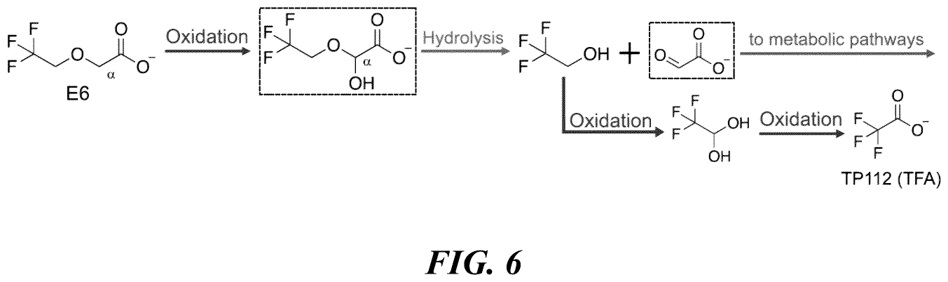

The alcohol product can be further oxidized, as spontaneous defluorination can occur with fluorine substitutions are present at the alcohol carbon, such as —CF₂OH as shown in FIG. 2I. The fluoroalkyl structure is unstable and can undergo spontaneous HF elimination followed by hydrolysis, which can lead to the cleavage of each C—F bond on the alcohol carbon as shown in FIG. 2I and FIGS. 5A and 5B. This instability accounts for why defluorination was observed for EE1, E4, E5, and E7 but not for E6, which was converted into an alcohol intermediate with a —CH₂OH moiety instead of a —CF₂OH moiety. Further oxidation of the alcohol intermediate of E6 (2,2,2-trifluoroethanol) led to the formation of trifluoroacetate (TFA) which was resistant to biodegradation and accumulated as an end product, as shown in FIG. 2F and FIG. 6. TFA was a major end product from E7 after the spontaneous defluorination, as only the C—F bonds next to the ether bond spontaneously cleaved. This was detected at the stoichiometric concentration, 37.2 μM, corresponding to the 37.9 μM removal of E7 and approximately 67 μM of fluorine ions at the end of the incubation as shown in FIGS. 2G and 2I.

The monooxygenation was faster for C4 structures such as E5-E7 than it was for C3 structures such as E1 and E4. The methyl branch in E4 led to a slower overall defluorination. The parent compound removal of E4 (39.3%) was similar to that of E1 (40.9%), which indicated that the hydroxylation of the parent compound was not impacted by the methyl branch. The lower level of defluorination of E4 in comparison to E1 was likely due to a slower dismutation of the intermediate caused by the steric effect of the methyl group, rendering a slower release of fluoride.

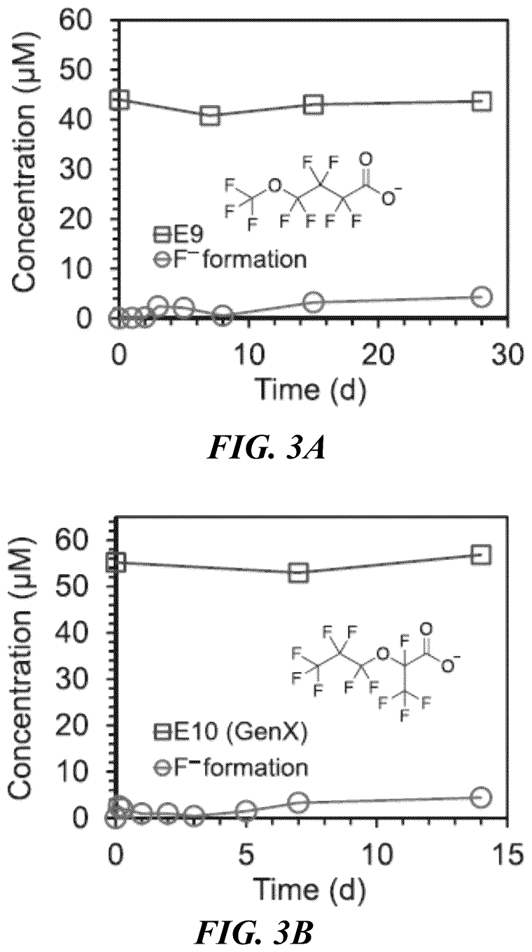

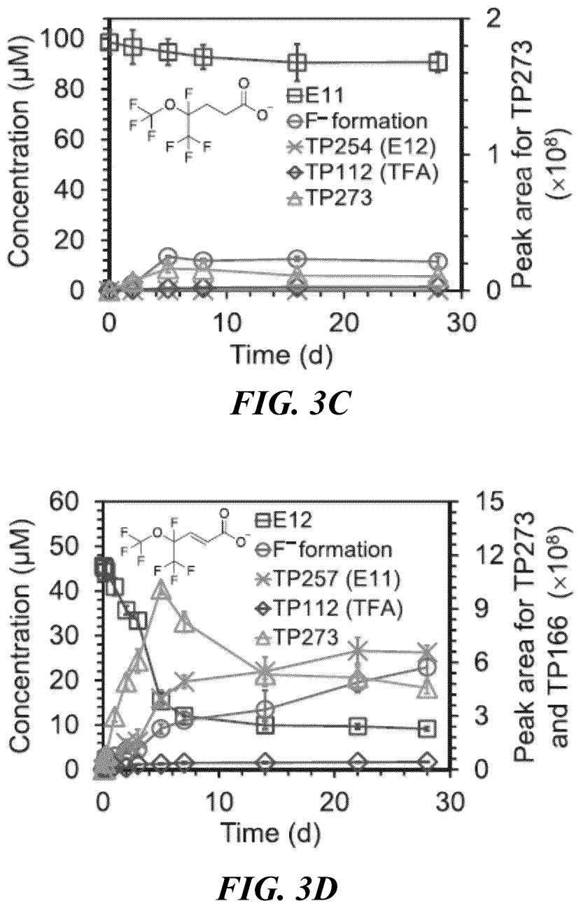

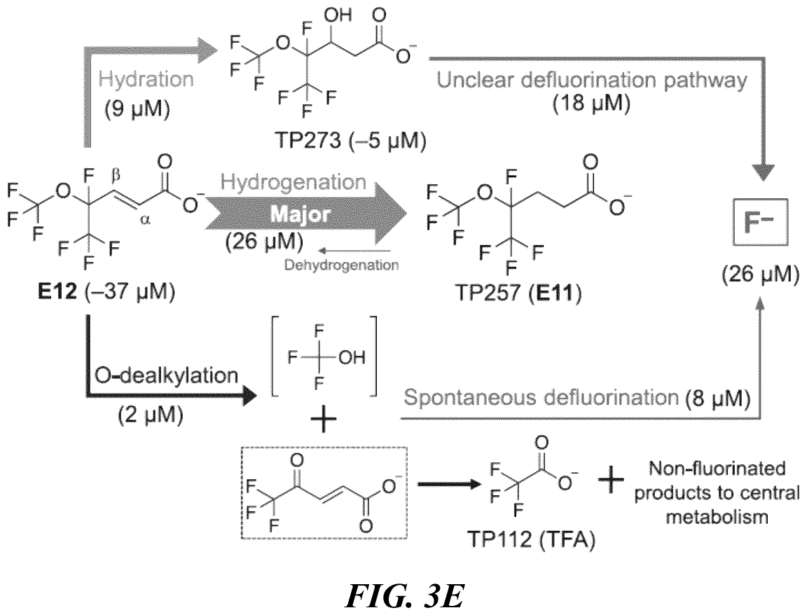

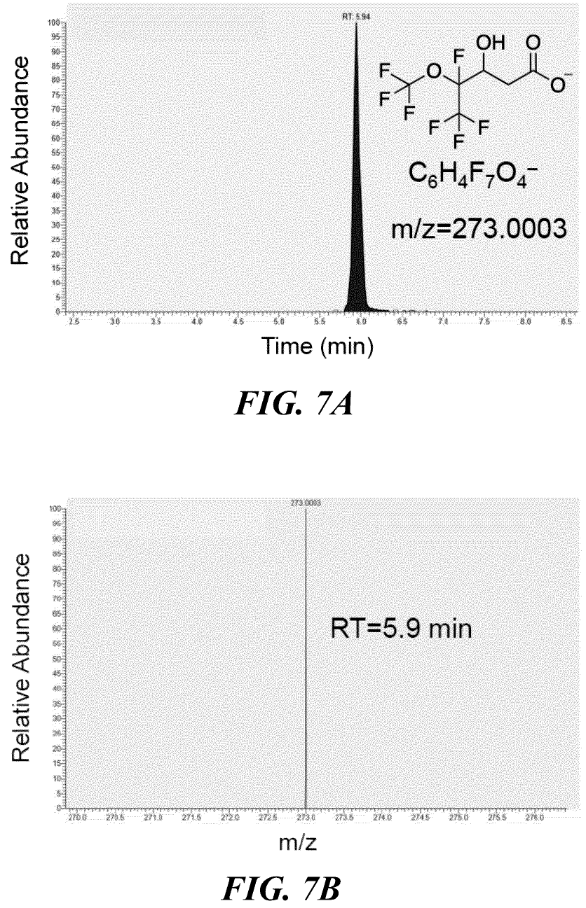

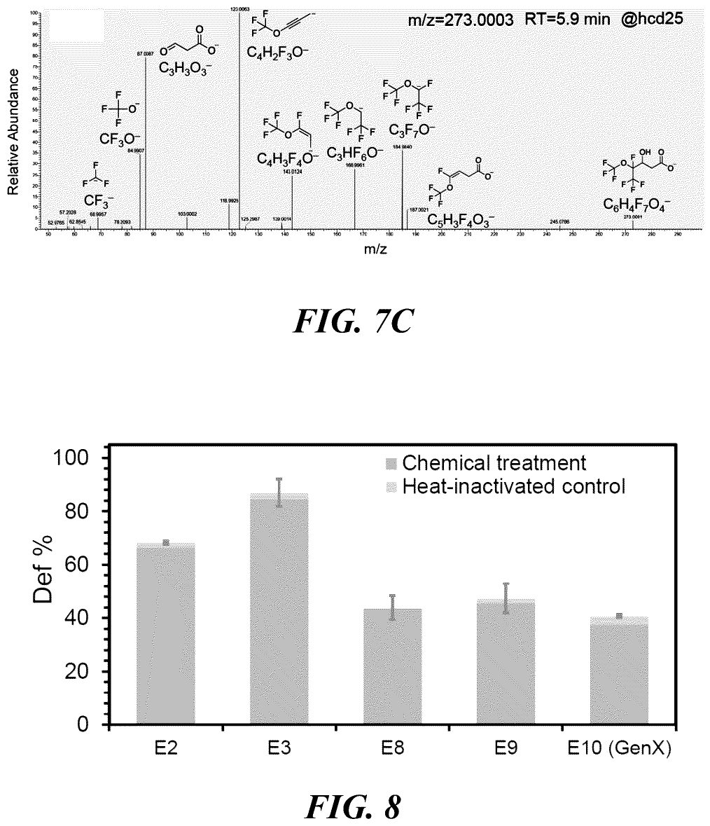

For the longer-chain ether PFAS such as E9-E12, the two perfluorinated structures, E9 and E10, did not show any biotransformation, as shown in FIGS. 3A and 3B. In contrast, the other two polyfluorinated structures, E11 and E12, exhibited biotransformation and defluorination but to a different extent, as shown in FIGS. 3C and 3D. The key difference between E11 and E12 is the saturation/unsaturation at the α-β position. However, with an unsaturated C═C bond, E12 exhibited 80% removal, whereas the removal of the saturated E11 was limited to 8%. This indicated that the C═C bond in E12 was more bioreactive than the C—C bond contained in E11. A majority of the removed E12, about 26 μM of the total 37 μM, underwent hydrogenation, which formed E11 within a week. The back transformation of E11 to E12 was slower, rendering a low removal of E11. In addition to the nondefluorinating hydrogenation pathway, E12 (11 μM) underwent two defluorinating pathways, determined through the two identified transformation products TP273 and TFA, shown in FIGS. 3C-3E and FIGS. 7A-7C. Of the 11 μM of E12, about 2 μM was converted into TFA, potentially through hydrolytic O-dealkylation, corresponding to the formation of 8 μM fluoride, four atoms of fluorine released per TFA formed, as shown in FIG. 3E. The remaining 9 μM of E12 was hydrated at the C═C bond, which formed TP273. The TP273 was further transformed, likely at the ether carbon, although the mechanism remains unclear, as shown in FIGS. 3D and 3E. This led to continuous defluorination after the depletion of E12 and the release of 18 μM of fluoride corresponding to about 5 μM of TP273 removal, approximately four atoms of fluorine cleaved from each TP273 molecule, as shown in FIG. 3E.

The ether bond played a critical role in the defluorination of E11 and E12 as the respective structures without the ether bond, i.e., FTMePA [(CF₃)₂CFCH₂COOH] and FTMeUPA [(CF₃)₂CFCH═CHCOOH], did not exhibit any defluorination in the activated sludge community taken from the same wastewater treatment plant (WWTP). FTMePA was resistant to biotransformation, while FTMeUPA underwent only hydrogenation forming FTMePA. Although the major biotransformation route for E12 was also hydrogenation, the ether bond enabled alternative routes that led to up to four fluorine ions being released per molecule of removed E12.

### Enhanced Defluorination of Recalcitrant Ether PFAS by the Chemical-Biological Treatment Train System

The highly and fully fluorinated ether PFAS, such as GenX and similar structures, was much more recalcitrant to biodegradation. Even chemical reductive degradation using UV (254 nm) and SO₃²⁻ (10 mM) at pH 9.5 showed sluggish defluorination, less than 50% after 48 h for GenX. The sluggish defluorination was due to the fluorine to hydrogen exchange products becoming more difficult to reduce. A combination with advanced chemical oxidation was implemented to achieve a greater level of defluorination. Less fluorinated products with more C—H bonds could be further transformed and defluorinated by aerobic microbial communities.

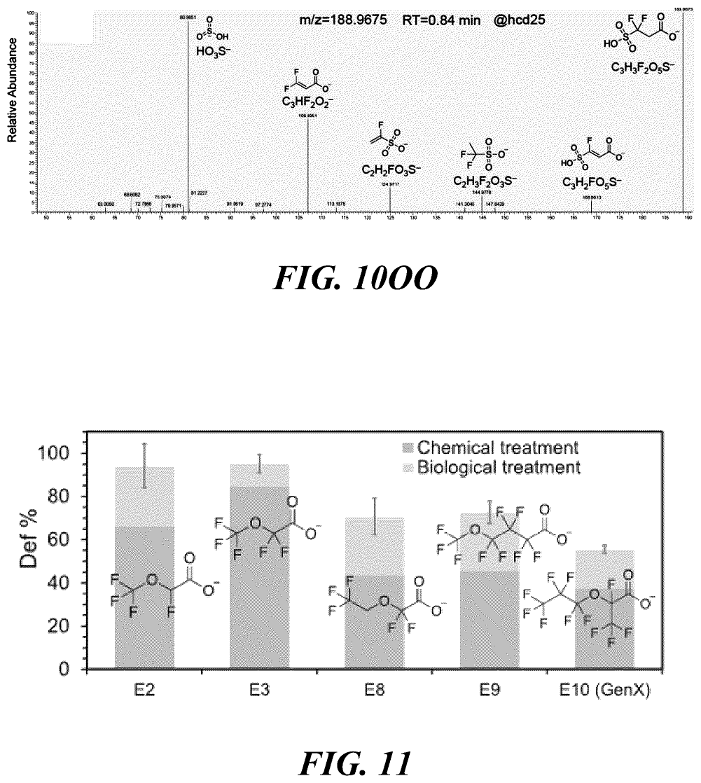

To achieve this degradation, utilizing aerobic microbial communities is more cost-effective than chemical oxidation. As proof of concept, the same activated sludge community was used to treat the effluent from a UV/SO₃²⁻ system treating all five nonbiodegradable ether PFAS, including E10 (GenX), E2, E3, E8, AND E9. An additional 11-28% defluorination was achieved by the aerobic biological post-treatment after eight days for all five compounds while no fluorine release was detected in the abiotic controls, as shown in FIGS. 8 and 11.

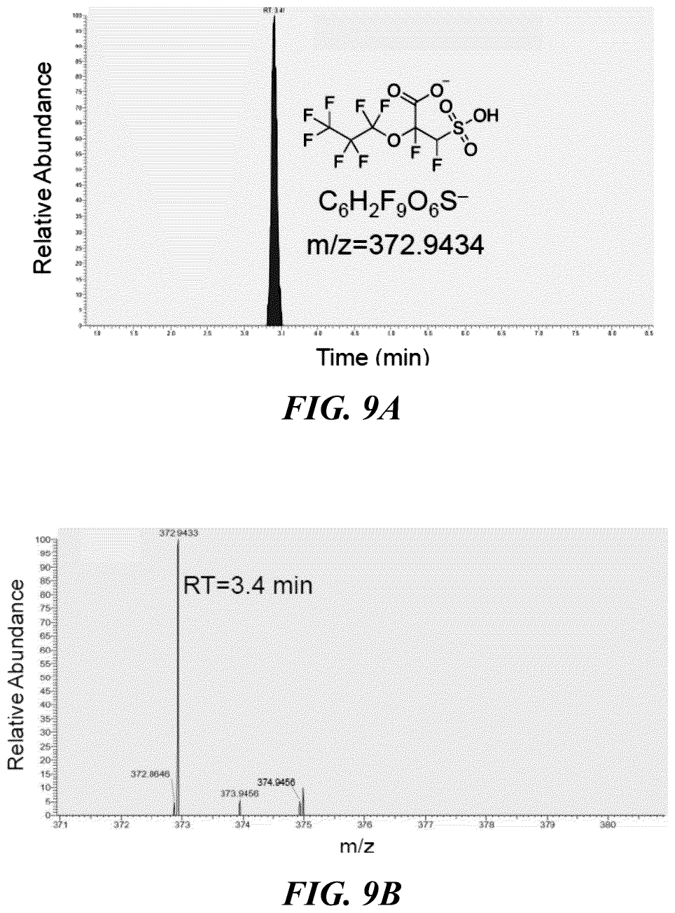

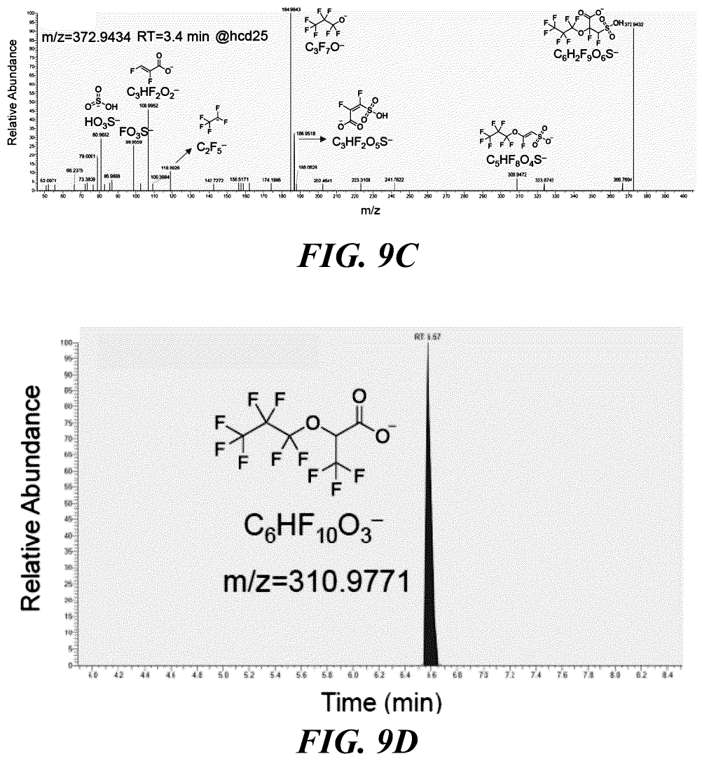

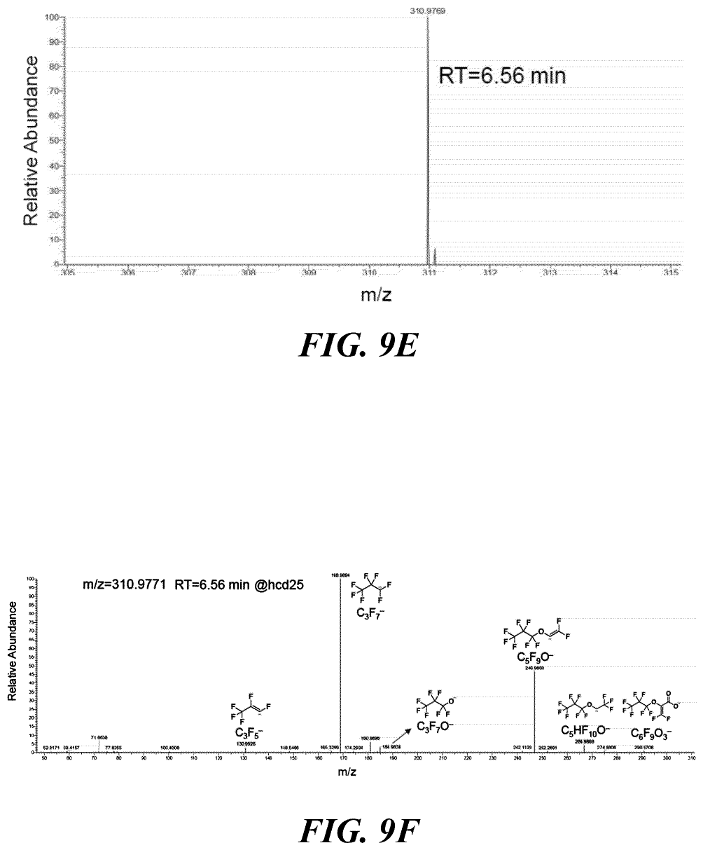

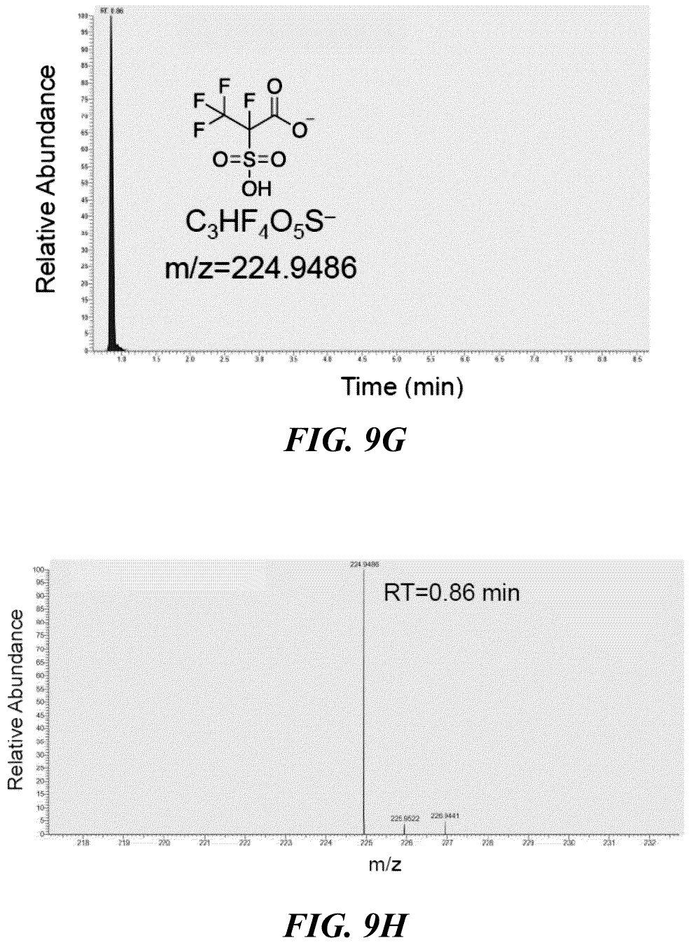

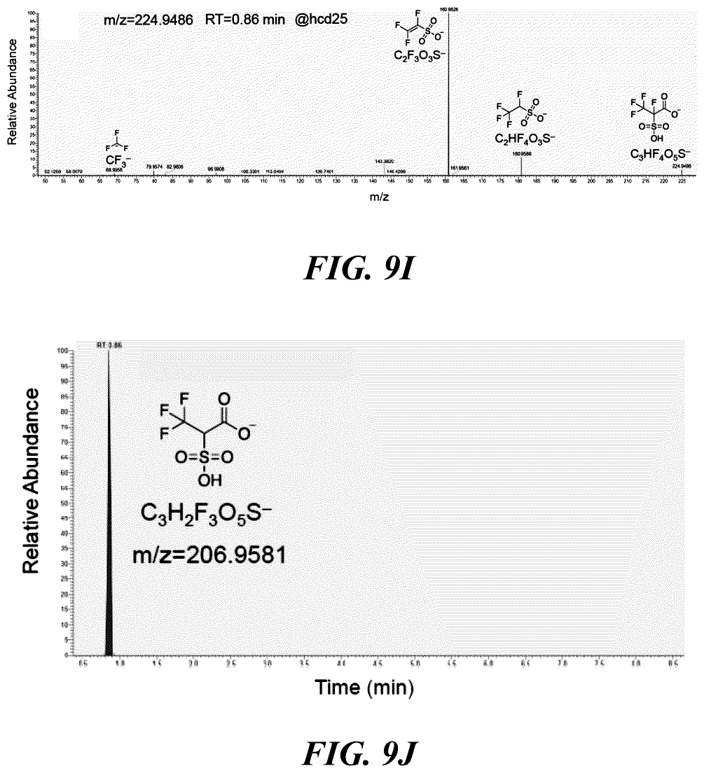

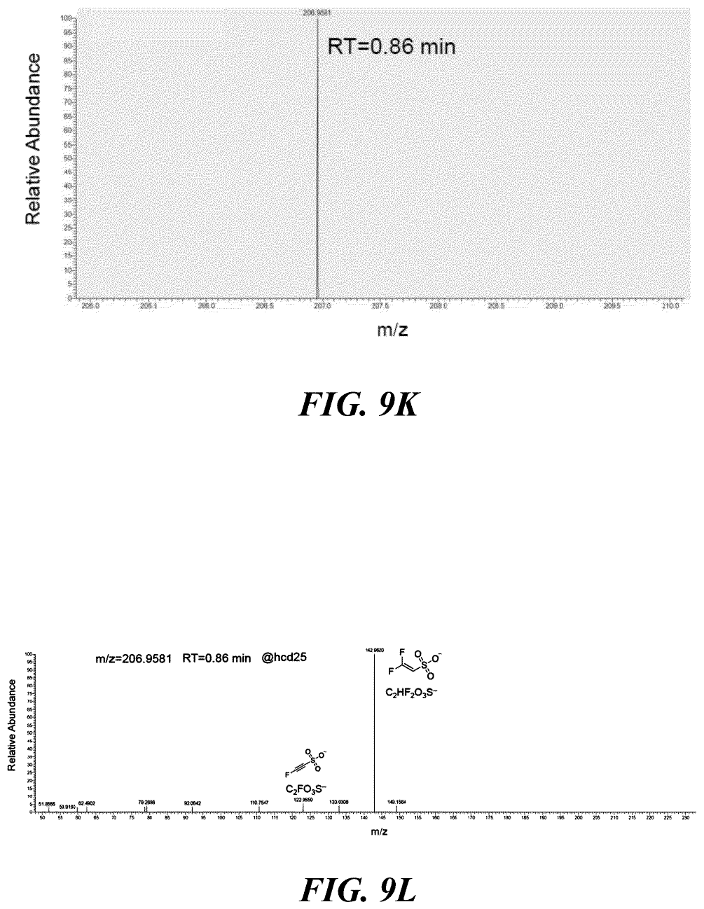

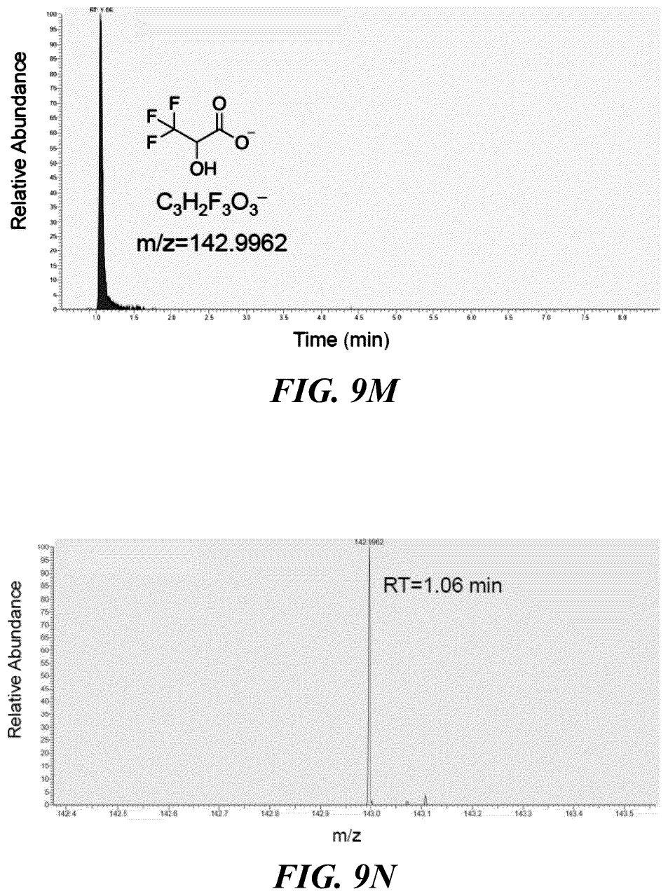

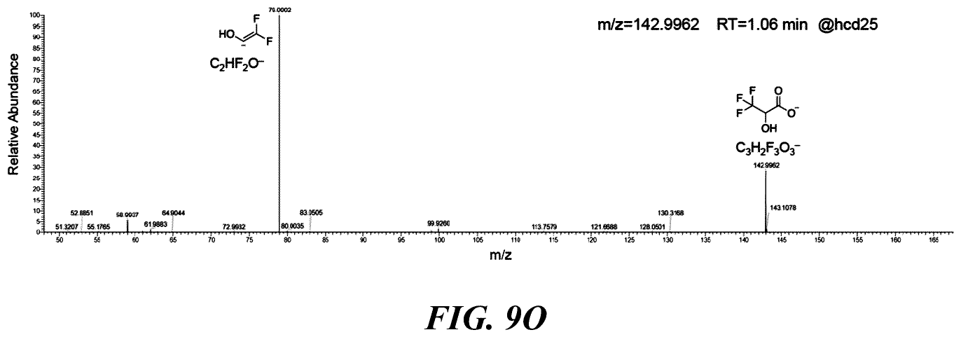

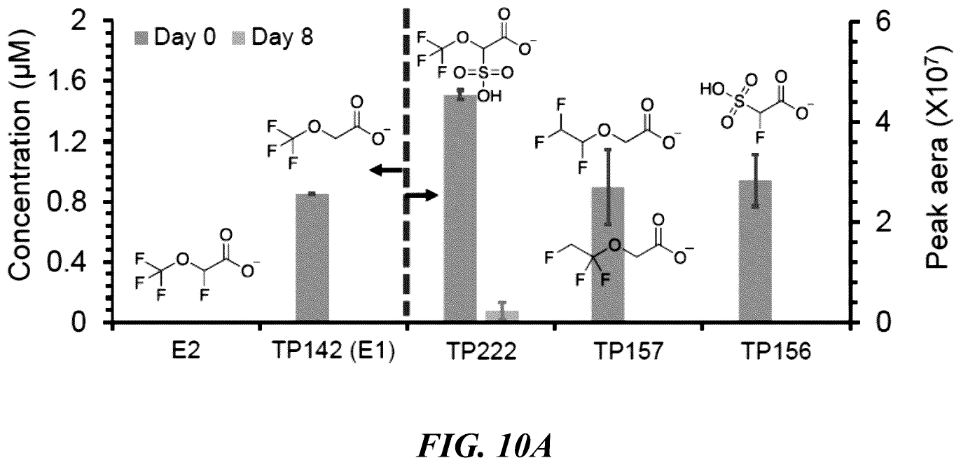

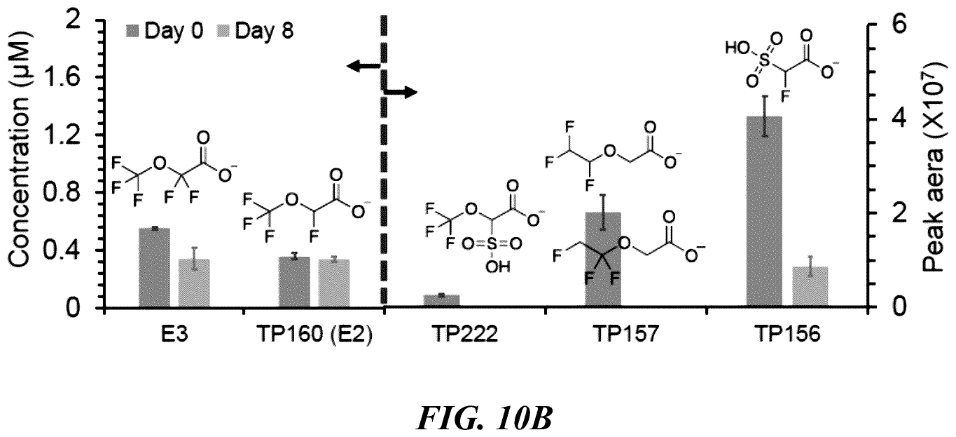

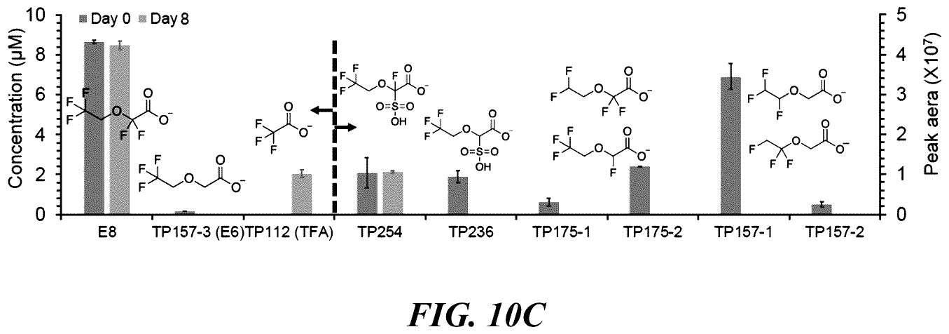

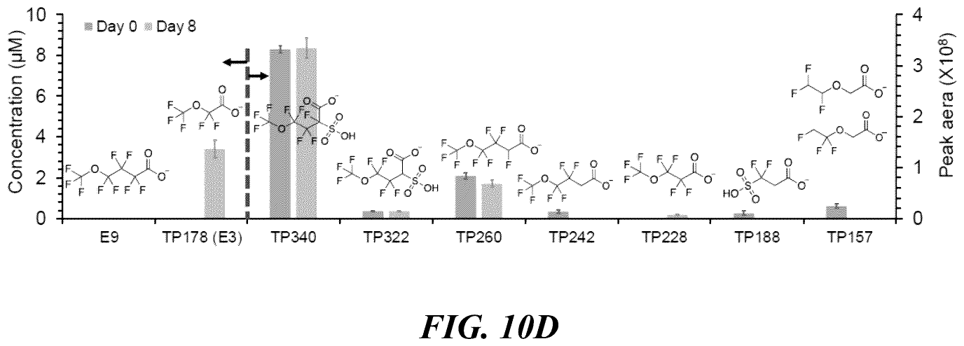

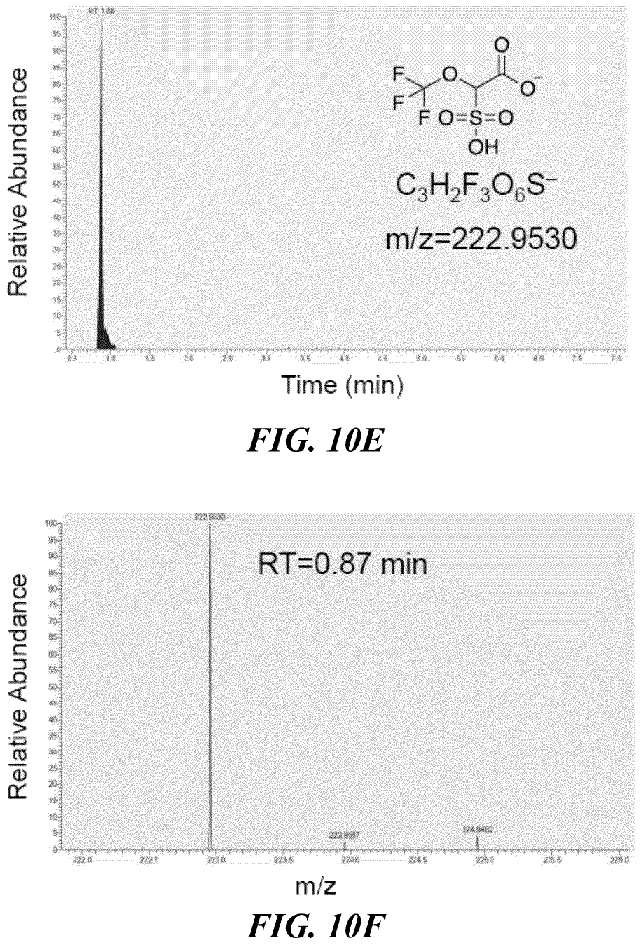

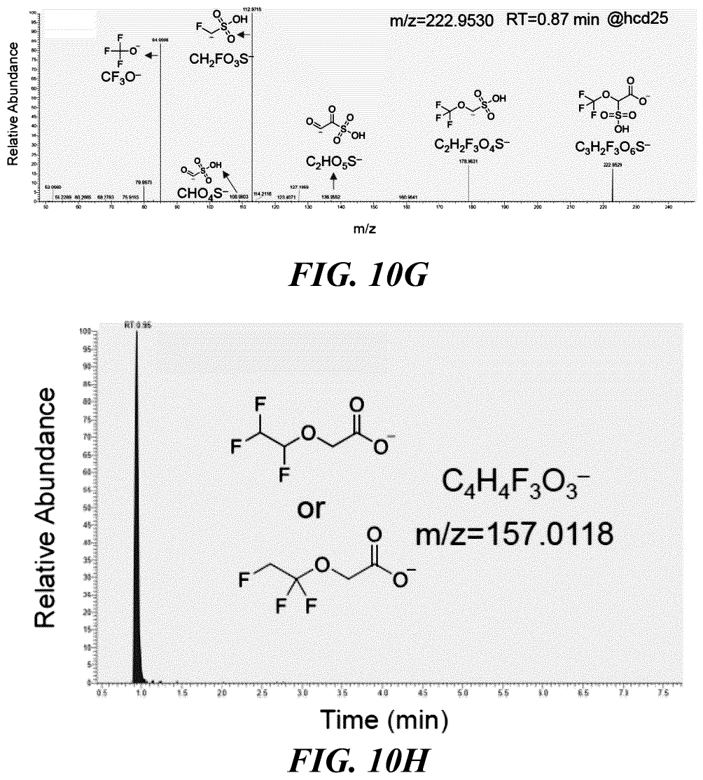

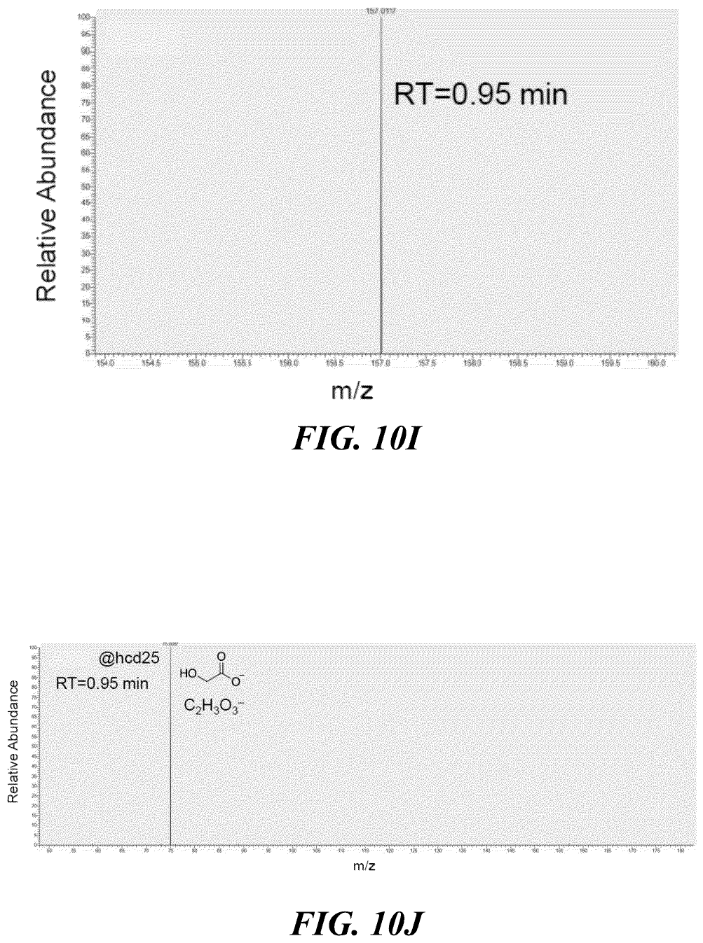

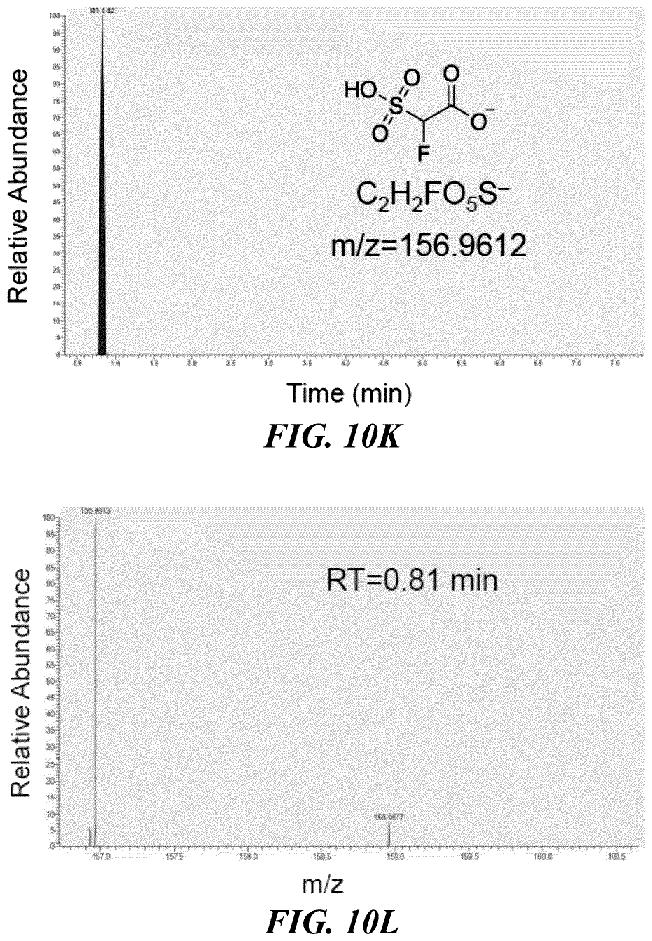

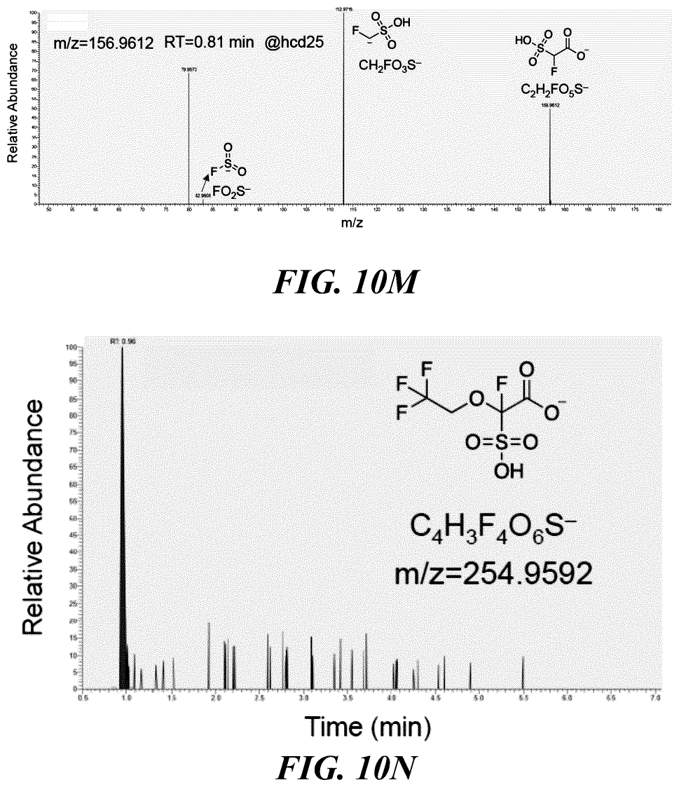

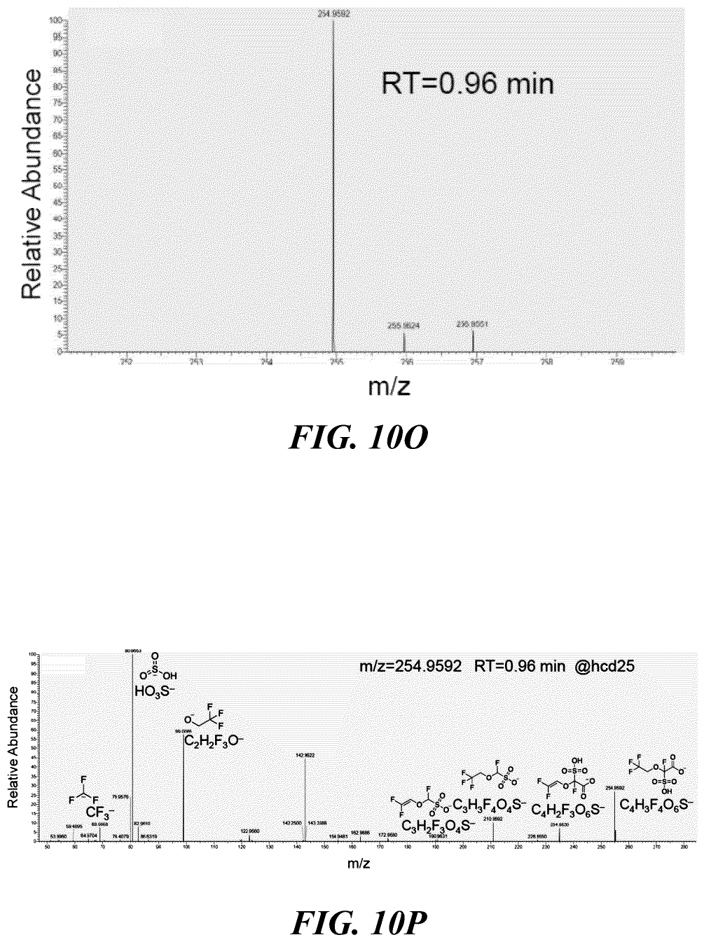

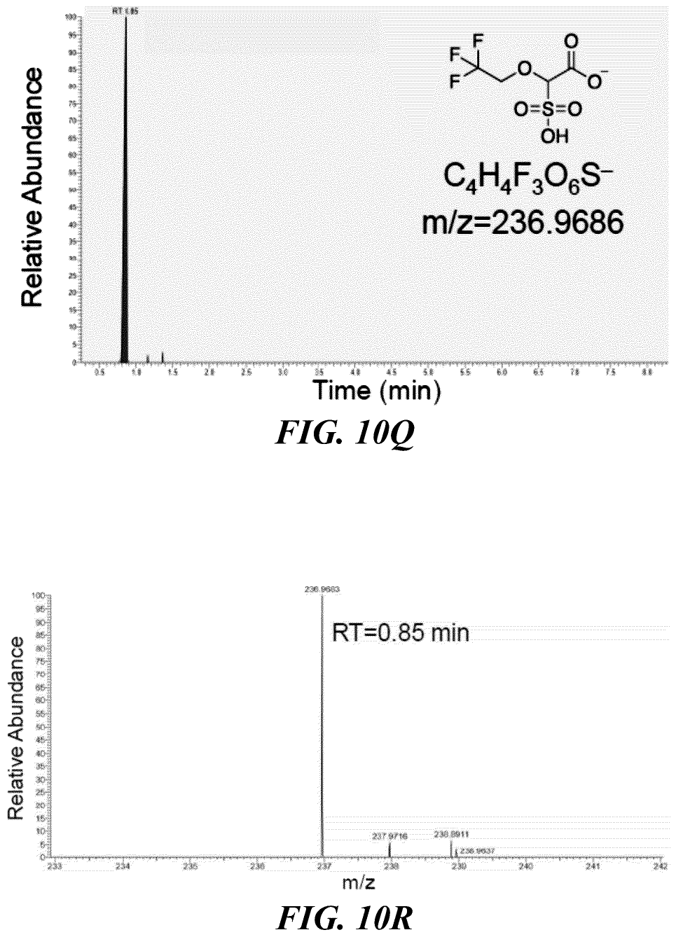

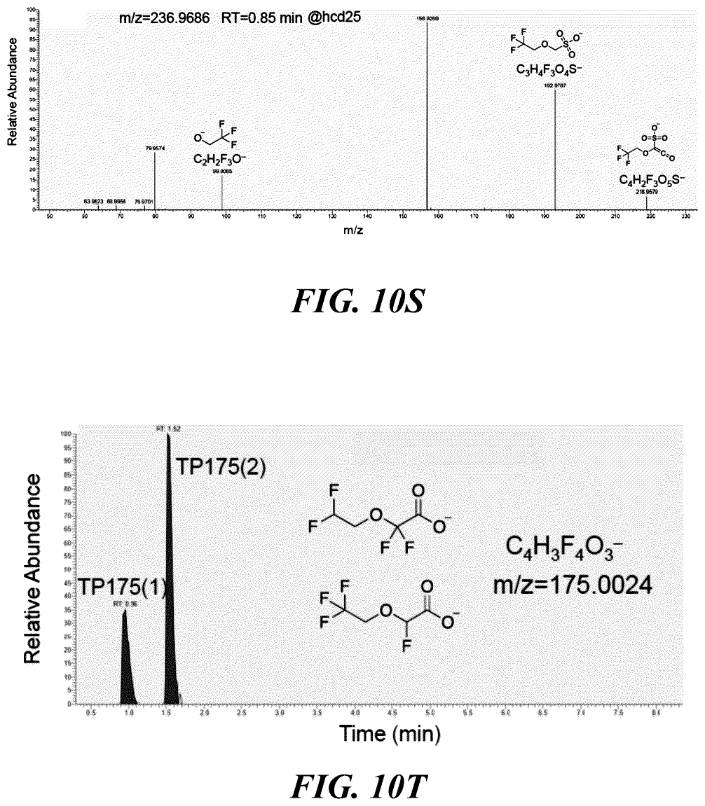

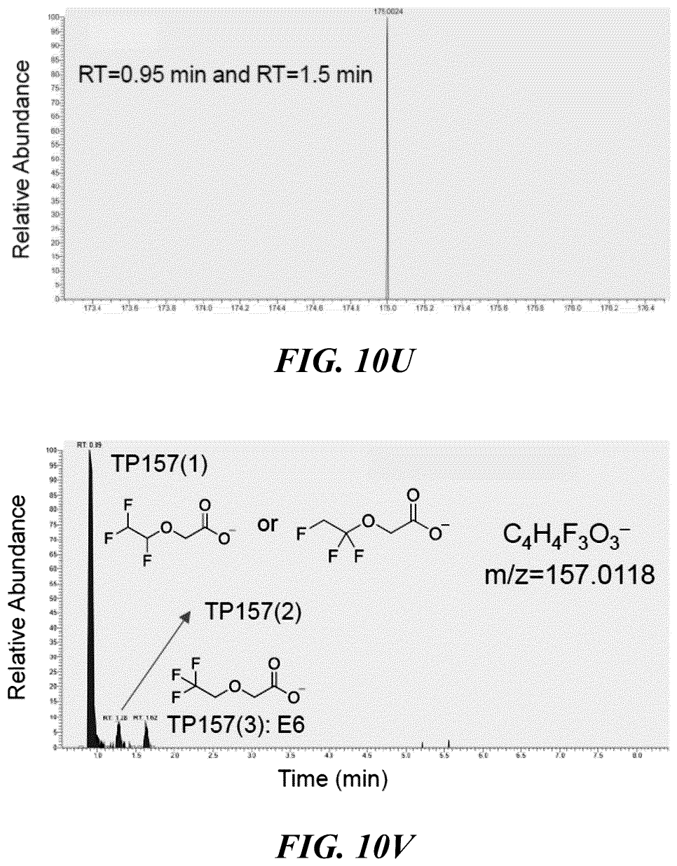

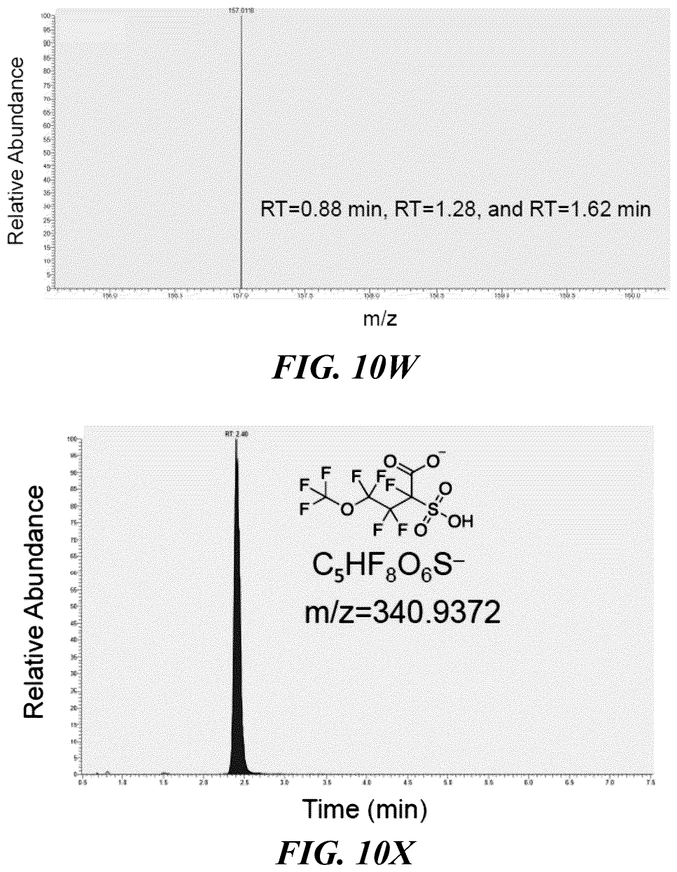

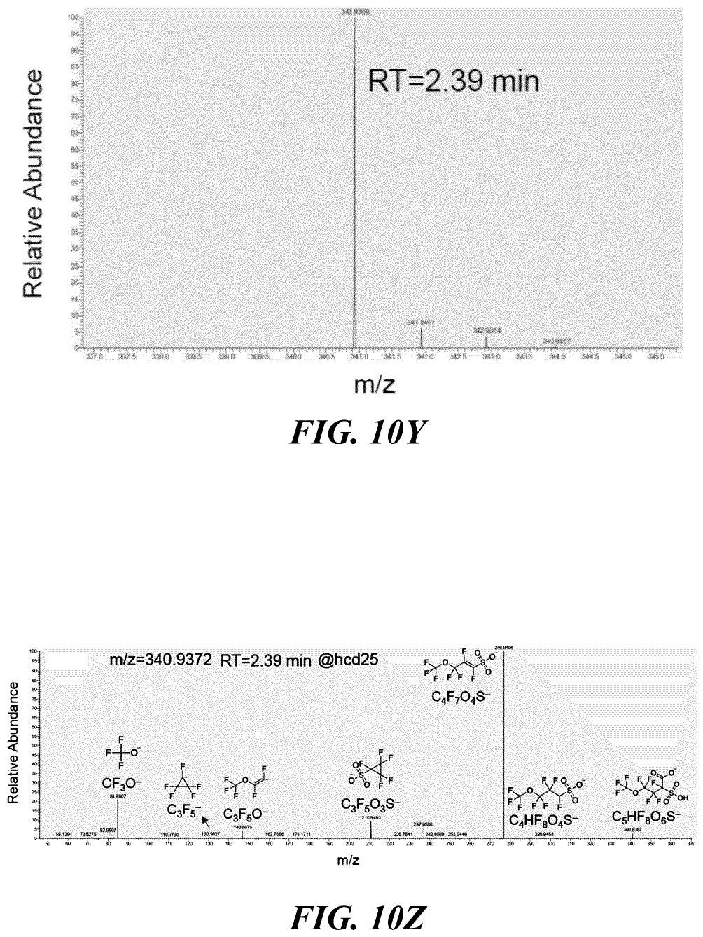

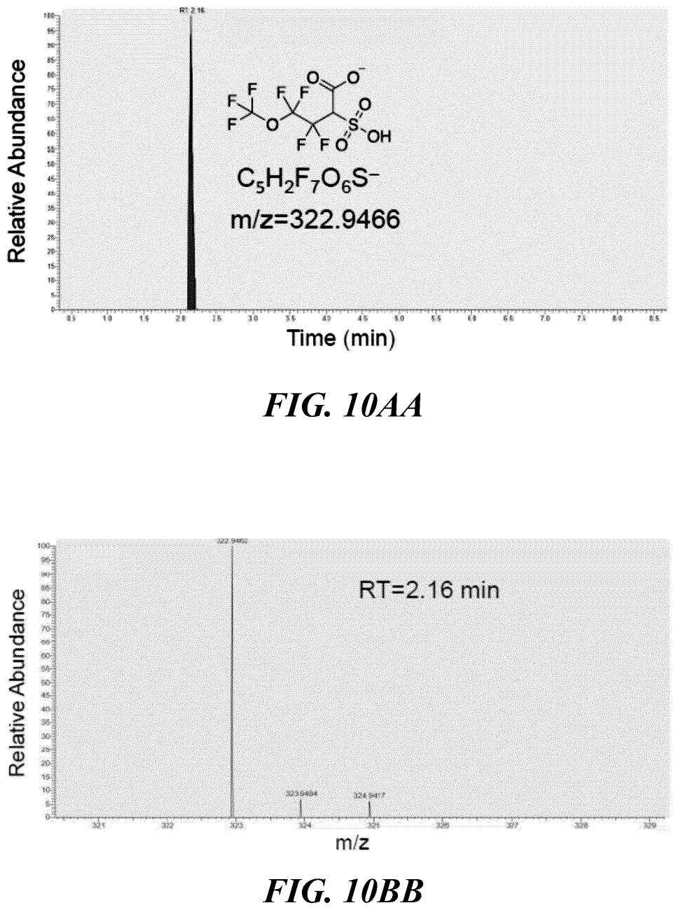

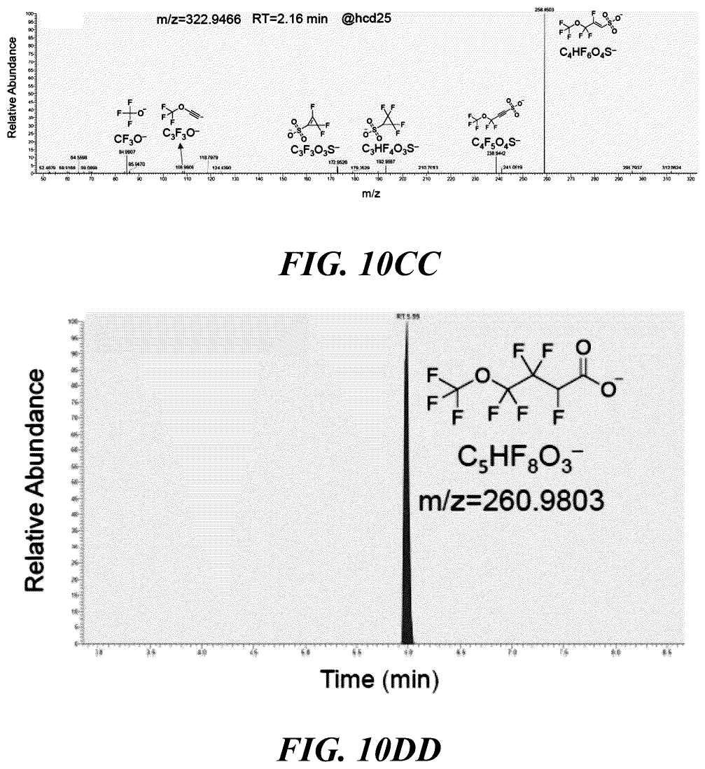

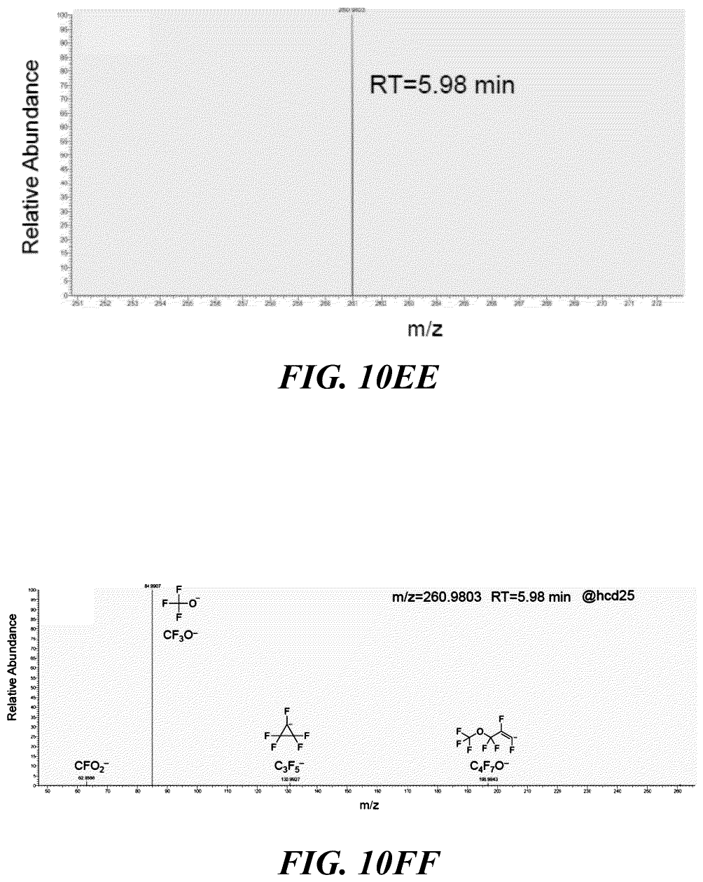

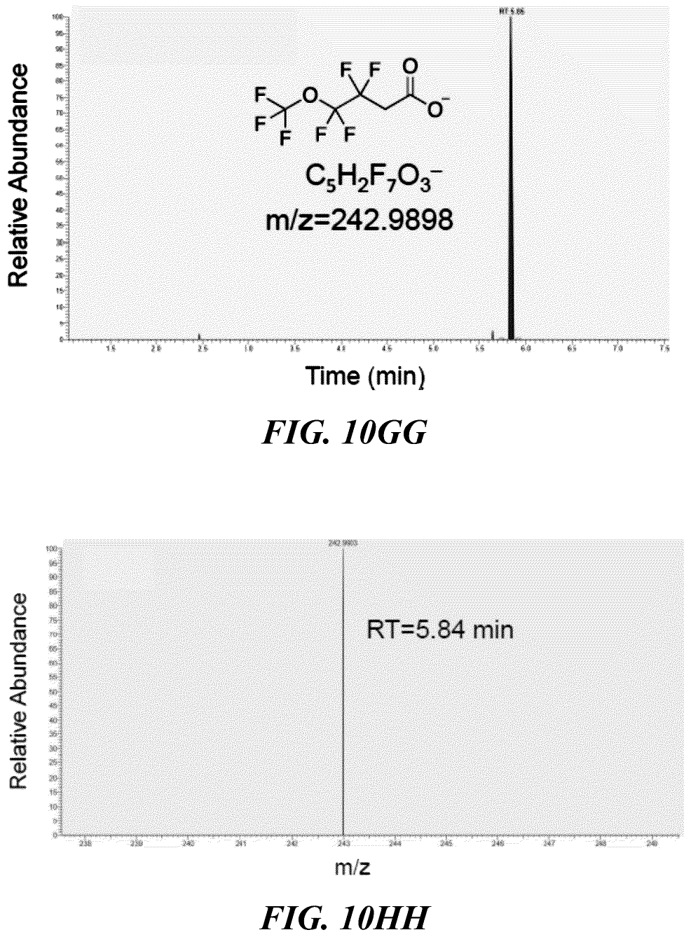

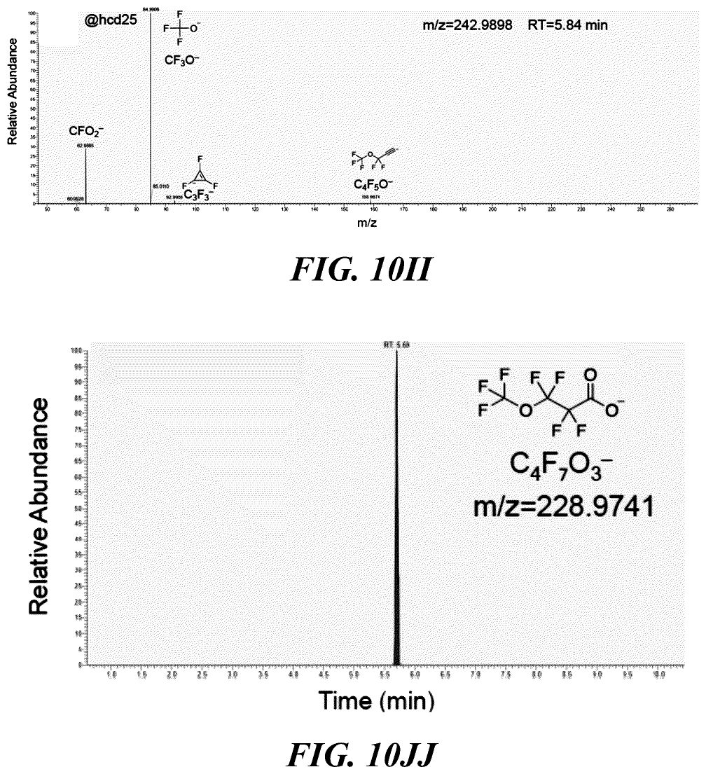

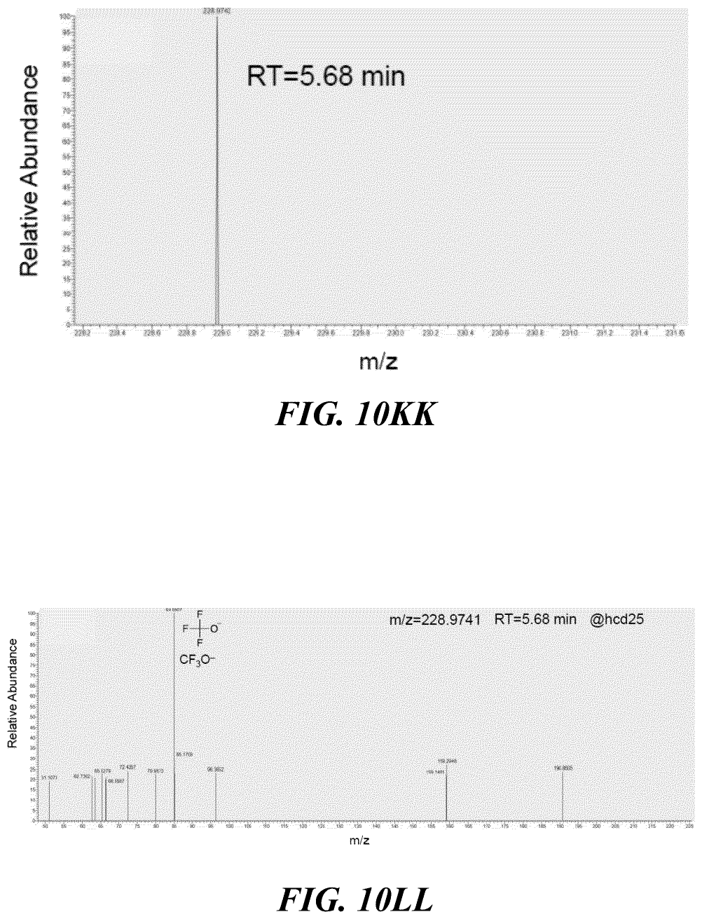

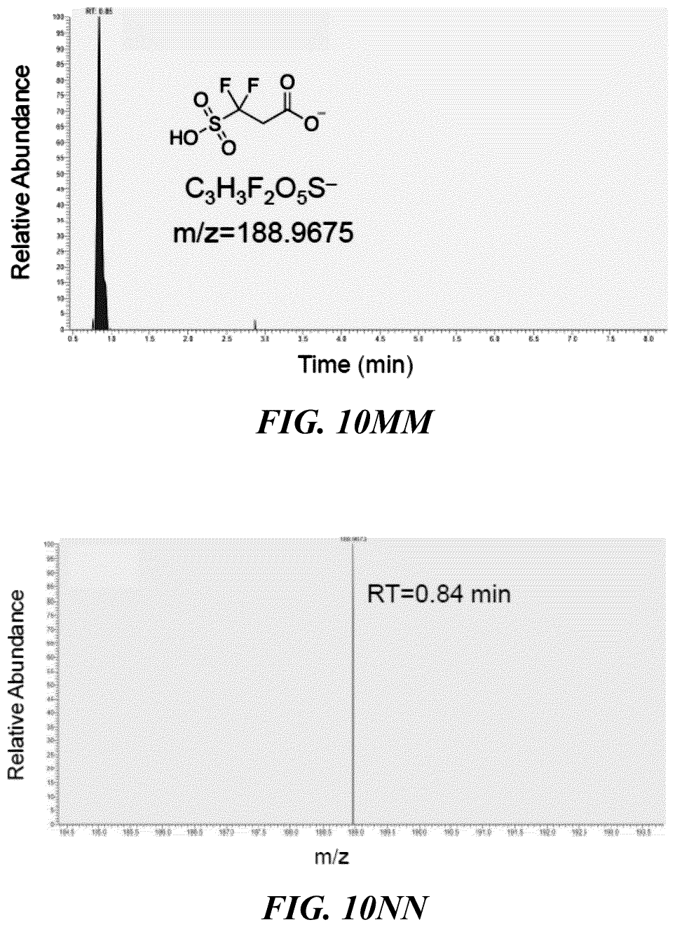

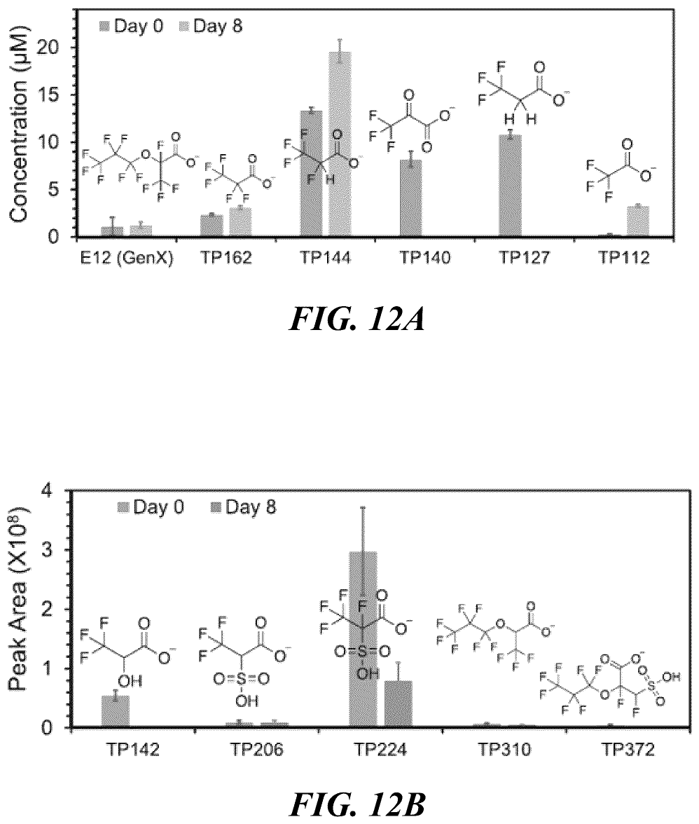

The additional defluorination mainly came from the aerobic biotransformation of chemical treatment products that contain the —CH₂— moiety next to the ether or carboxylic group, as shown in FIGS. 9A-9O, FIGS. 10A-10OO, and FIGS. 12A-12B. With GenX as an example, in addition to trifluoropropionate (TP127), which is completely defluorinated in activated sludge communities, the sulfonated chemical treatment products TP224 and TP372 were also microbially transformed via desulfonation or hydroxylation, forming unstable fluoroalcohol intermediates, hence contributing to the additional defluorination as shown in FIG. 12B. The slight defluorination PFPrA (TP162) appeared to be the result of the O-dealkylation of TP372 as shown in FIGS. 12A and 12B. In addition, trifluoroacetate (TFA) was formed via the nondefluorinating biotransformation of certain chemical treatment products such as trifluoropyruvate (TP140), trifluoroacetate (TP142), and sulfonated trifluoropropionate (TP206).

### Environmental Implications

As shown in this experiment, ether bonds in PFAS molecules can serve as a weak point for aerobic microorganisms to attack, playing critical roles in enhancing biodegradability and defluorination potential. At least one —CH₂— moiety on the nonfluorinated side of the —O— bond was necessary to trigger an active microbial attack. Monooxygenation occurred at the —CH₂— moiety next to the —O— bond, forming an unstable hemiacetal intermediate, which underwent O-dealkylation forming an alcohol and an aldehyde. Spontaneous defluorination occurred to the alcohol product when the alcohol carbon was fluorinated, such as CF₃OH and —CF₂OH. Many microbial species possessing monooxygenases or cytochromes P-450 can cleave ether structures, either alkyl or aryl, through this oxidation pathway. A β-oxidation-like pathway for polyfluorocarboxylic acids (PFCAs), CF₃(CF₂)ₘ(CH₂)ₙCOOH, where the defluorination can be observed for odd numbers of n is known. Ether bonds in PFCA structures, such as CF₃(CF₂)ₘO(CH₂)ₙCOOH, undergo defluorination at the carbon next to the ether triggered by oxidative ether cleavage. Hydrolytic O-dealkylation was also observed for unsaturated ether PFAS structures that do not contain a —CH₂ moiety adjacent to the ether bond, such as E12. The C═C bond made the structure more biodegradable compared to its saturated counterpart. The ether bond in the structure further enabled defluorination triggered either by direct hydrolytic O-dealkylation or hydration at the C═C bond. Both reactions have been proposed for ether cleavage in nonfluorinated unsaturated ether structures with the double bond on the ether carbon, such as vinyl ether and isochorismic acid with a —O—C═C— moiety. The activated sludge community used in this experiment came from a typical municipal WWTP. Similar biotransformation activities have been observed in other municipal WWTP's. Microorganisms in activated sludge communities commonly occur in natural environments, where similar biotransformation kinetics can vary depending on the microbial activities and abundance. Understanding the role played by ether bonds in the biodegradability of ether PFAS structures and the transformation mechanisms, particularly the structure-biodegradability relationship, can help predict the environmental fate of various ether PFAS structures that are being used and discharged into natural and engineered environments. It can also provide important guidance for the design of biodegradable alternative PFAS.

The chemical-biological waste treatment train system achieved enhanced destruction of ether PFAS that cannot be effectively treated by either method alone. Perfluorinated ether PFAS are resistant to advanced oxidation by hydroxyl radicals. Advanced reduction by hydrated electrons reached only <50% total defluorination for GenX and its derivatives due to the formation of more C—H bonds via the undesirable reductive defluorination pathway. Nevertheless, what is undesirable in chemical treatment is attractive to microorganisms. An approximate 28% increase in the total defluorination was achieved by aerobic biological post-treatment, where many chemical treatment products with a —CH₂— moiety were further biodegraded and defluorinated into short-chain perfluorinated acids. Compared to advanced oxidation processes (AOPs) which would result in the same short-chain perfluorinated end products, the biological post-treatment was more cost-effective, particularly when there was more high-level nonfluorinated dissolved organic carbon in the matrix that can impact the AOP performance. After the aerobic biological post-treatment, although the defluorination of GenX was still incomplete, it simplified the major end products to three short-chain PFCAs, PFPrA, TFA, and 2,3,3,3-tetrafluoropropionic acid. The three major end products can then be further degraded through a second advanced reduction treatment. TFA can be quickly and completely degraded by UV/sulfite treatment, while PFPrA and 2,3,3,3-tetrafluoropropionic acid may form the chemically stable trifluoropropionate, which can be completely destroyed in another round of aerobic biological post-treatment. Upon implementation of appropriate recirculation, the chemical-biological treatment train system could achieve nearly complete destruction of GenX and other structurally similar ether PFAS that show no or low-level destruction in biological and chemical treatment alone.

### Example 2

### Chemical and Biological Degradation of Ether PFAS

The first treatment unit using advanced reduction partially defluorinated GenX and similar highly fluorinated ether PFAS. The total defluorination of GenX and the other medium chain structures (n≥4) was lower than about 50%, as shown in FIG. 11. Less fluorinated products with more C—H bonds were formed in this degradation, as shown in FIGS. 12A and 12B. The incomplete degradation products were further transformed and defluorinated by the aerobic biological post-treatment. An additional 11%-28% defluorination was achieved after eight days for each of the five tested ether PFAS compounds as shown in FIG. 11. For GenX, the chemical treatment products can be converted into at least three major products after the biological post-treatment process: PFPrA, TFA, and 2-H polyfluoropropionic acid.

With an internal recycle between the chemical and biological units, effluent of the biological unit containing PFPrA, TFA, and 2-H polyfluoropropionic acid can be returned to further break down the three products. During an internal recycle, TFA was fully degraded, and PFPrA and 2-H polyfluoropropionic acid were converted and mineralized as shown in FIGS. 12A and 12B.

Various embodiments of the present technology are set forth herein below in paragraphs [0160]-[0187]:

Embodiment 1. A method of chemically and biologically degrading ether per- and polyfluoroalkyl substance (PFAS) molecules, the method comprising:
depositing wastewater into a chemical treatment unit, wherein the chemical treatment unit contains one or more UV lamps and a sulfite solution;
retaining the wastewater in the chemical treatment unit for a first time period;
transferring the wastewater from the chemical treatment unit to a biological treatment unit, wherein the biological treatment unit contains a sludge community in aerobic conditions;
retaining the wastewater in the biological treatment unit for a second time period; and
testing the wastewater to determine the concentration of chemical compounds selected from the group consisting of ether PFAS and/or breakdown products from the biological degradation of ether PFAS compounds.

Embodiment 2.The method of embodiment 1, wherein the sulfite solution is generated from one or more of sodium sulfite and potassium sulfite.

Embodiment 3. The method of embodiment 1, wherein the concentration of the sulfite solution is between about 10.0 mM and about 20.0 mM.

Embodiment 4. The method of embodiment 1, wherein the concentration of the sulfite solution is approximately 10.0 mM.

Embodiment 5. The method of embodiment 1, wherein the one or more UV lamps emit UV radiation at approximately 254 nm.

Embodiment 6. The method of embodiment 1, wherein the sludge community is combined with an activated sludge bacterial community taken from a wastewater treatment plant.

Embodiment 7. The method of embodiment 1, wherein the sludge community is an activated sludge community.

Embodiment 8. The method of embodiment 1, wherein the sludge community is taken from a wastewater treatment plant.

Embodiment 9. The method of embodiment 1, wherein the first time period is between about 24 hours to about 48 hours.

Embodiment 10. The method of embodiment 1, wherein the second time period is between about 5 days to about 15 days.

Embodiment 11. The method of embodiment 1, wherein the second time period is about 8 days.

Embodiment 12. The method of embodiment 1, additionally comprising the steps:
transferring the wastewater into the chemical treatment unit from the biological treatment unit;
retaining the wastewater in the chemical treatment unit for a third time period;
transferring the wastewater from the chemical treatment unit to the biological treatment unit; and
retaining the wastewater in the biological treatment unit for a fourth time period.

Embodiment 13. The method of embodiment 12, wherein the third time period is between about 24 hours to about 48 hours.

Embodiment 14. The method of embodiment 12, wherein the fourth time period is between about 5 days to about 15 days.

Embodiment 15. The method of embodiment 12, wherein the fourth time period is about 8 days.

Embodiment 16. The method of embodiment 1, wherein the biological treatment unit is inoculated with one or more bacteria.

Embodiment 17. The method of embodiment 1, wherein the biological treatment unit is inoculated with a cell culture containing one or more bacteria.

Embodiment 18. The method of embodiment 17, wherein the biological treatment unit is inoculated with an autoclaved spent media of the cell culture containing one or more bacteria.

Embodiment 19. The method of embodiment 1, wherein the biological treatment unit is inoculated with a cell culture containing the one or more bacteria and an autoclaved spent media of the cell culture containing the one or more bacteria.

Embodiment 20. The method of embodiment 1, wherein the chemical treatment unit and the biological treatment unit are connected in series.

Embodiment 21. The method of embodiment 1, wherein the chemical treatment unit and the biological treatment unit are separate.

Embodiment 22. The method of embodiment 1, wherein the chemical treatment unit and/or the biological treatment unit are linked to a wastewater outlet flow from a commercial or industrial site.

Embodiment 23. The method of embodiment 1, wherein the chemical treatment unit and/or the biological treatment unit are linked to a wastewater treatment plant.

Embodiment 24. The method of embodiment 1, wherein the chemical treatment unit and/or the biological treatment unit are linked to a wastewater flow.

Embodiment 25. The method of embodiment 1, wherein the chemical treatment unit and/or the biological treatment unit contain a chemical monitoring system.

Embodiment 26. The method of embodiment 1, wherein the wastewater contained within the chemical treatment unit or the biological treatment unit is tested for residual ether PFAS and degradation byproducts through automated sample removal and/or sample testing.

Embodiment 27. The method of embodiment 1, wherein the wastewater contained within the chemical treatment unit or the biological treatment unit is tested for residual ether PFAS and degradation byproducts through manual sample removal and/or sample testing.

Embodiment 28. A system or apparatus to implement a method recited in any of embodiments 1 to 27.

While this specification contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few implementations are disclosed. However, variations and enhancements of the disclosed implementations and other implementations can be made based on what is described and illustrated in this specification.

From the foregoing, it will be appreciated that specific embodiments of the invention have been described herein for purposes of illustration, but that various modifications may be made without deviating from the scope of the invention. Accordingly, the invention is not limited except as by the appended claims.

We claim:

1. A method of chemically and biologically degrading ether per-and polyfluoroalkyl substance (PFAS) molecules, the method comprising:
depositing wastewater into a chemical treatment unit, wherein the chemical treatment unit contains one or more UV lamps and a sulfite solution;
retaining the wastewater in the chemical treatment unit for a first time period;
transferring the wastewater from the chemical treatment unit to a biological treatment unit, wherein the biological treatment unit contains a sludge community in aerobic conditions;
retaining the wastewater in the biological treatment unit for a second time period; and
testing the wastewater to determine a concentration of chemical compounds selected from the group consisting of ether PFAS and/or breakdown products from the biological degradation of ether PFAS compounds.

2. The method of claim 1, wherein the sulfite solution is generated from one or more of sodium sulfite and potassium sulfite.

3. The method of claim 1, wherein the concentration of the sulfite solution is between about 10.0 mM and about 20.0 mM.

4. The method of claim 1, wherein the one or more UV lamps emit UV radiation at approximately 254 nm.

5. The method of claim 1, wherein the sludge community is combined with an activated sludge bacterial community taken from a wastewater treatment plant.

6. The method of claim 1, wherein the sludge community is an activated sludge community.

7. The method of claim 1, wherein the sludge community is taken from a wastewater treatment plant.

8. The method of claim 1, wherein the first time period is between about 24 hours to about 48 hours, and the second time period is between about 5 days to about 15 days.

9. The method of claim 1, additionally comprising:
transferring the wastewater into the chemical treatment unit from the biological treatment unit;
retaining the wastewater in the chemical treatment unit for a third time period;
transferring the wastewater from the chemical treatment unit to the biological treatment unit; and
retaining the wastewater in the biological treatment unit for a fourth time period.

10. The method of claim 9, wherein the third time period is between about 24 hours to about 48 hours, and the fourth time period is between about 5 days to about 15 days.

11. The method of claim 1, wherein the biological treatment unit is inoculated with one or more bacteria.

12. The method of claim 1, wherein the biological treatment unit is inoculated with a cell culture containing one or more bacteria.

13. The method of claim 1, wherein the chemical treatment unit and the biological treatment unit are connected in series.

14. The method of claim 1, wherein the chemical treatment unit and the biological treatment unit are separate.

15. The method of claim 1, wherein the chemical treatment unit and/or the biological treatment unit are linked to a wastewater outlet flow from a commercial or industrial site.

16. The method of claim 1, wherein the chemical treatment unit and/or the biological treatment unit are linked to a wastewater treatment plant.

17. The method of claim 1, wherein the chemical treatment unit and/or the biological treatment unit are linked to a wastewater flow.

18. The method of claim 1, wherein the chemical treatment unit and/or the biological treatment unit contain a chemical monitoring system.

19. The method of claim 1, wherein the wastewater contained within the chemical treatment unit or the biological treatment unit is tested for residual ether PFAS and degradation byproducts through automated sample removal and/or sample testing.

20. The method of claim 1, wherein the wastewater contained within the chemical treatment unit or the biological treatment unit is tested for residual ether PFAS and degradation byproducts through manual sample removal and/or sample testing.
